(19) United States
(12) Patent Application Publication
(10) Pub. No.: US 2023/0293432 A1
(43) Pub. Date: Sep. 21, 2023

(54) POLYMERIC MICELLES COMPRISING GLUCURONIDE-PRODRUGS

(71) Applicant: Rheinisch-Westfälische Technische Hochschule (RWTH), Aachen (DE)

(72) Inventors: Yang SHI, Aachen (DE); Twan LAMMERS, Maaseik (BE); Josbert Maarten METSELAAR, Naarden (NL)

(21) Appl. No.: 18/014,274

(22) PCT Filed: Jul. 6, 2021
(86) PCT No.: PCT/EP2021/068694

§ 371 Date: Jan. 3, 2023

(30) Foreign Application Priority Data
Jul. 6, 2020 (DE) 10 2020 208 448.3

(51) Int. Cl. A61K 9/107; A61K 31/7068; A61K 31/704

(52) U.S. Cl. CPC A61K 9/1075; A61K 31/7068; A61K 31/704

(57) ABSTRACT

The invention relates to a polymeric micelle comprising a block copolymer comprising a polyethylene glycol (PEG) hydrophilic block and a hydrophobic block, and a compound according to formula (I) or formula (III) encapsulated within said polymeric micelle and to uses thereof.

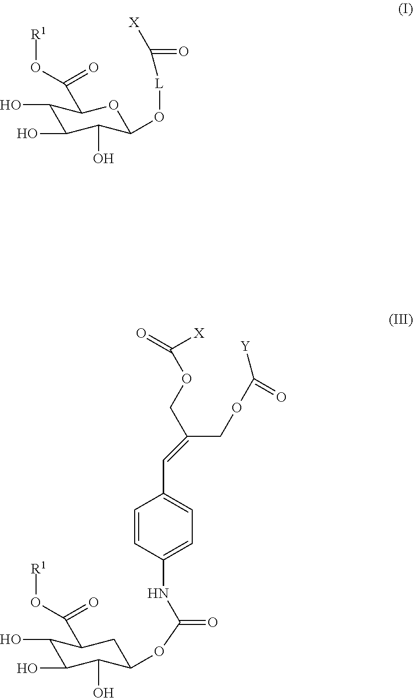

### FIELD OF THE INVENTION

The present invention relates to compositions of hydrophilic drug molecules, in particular anticancer, antifungal and/or antibiotic drugs, that enable targeted delivery of the drugs to tumors and sites of infection. In particular, the present invention relates to compositions comprising polymeric micelles and encapsulated hydrophilic drug molecules modified with glucuronide using a spacer molecule that promotes physical interaction, entrapment and stabilization of the drug-loaded micelles.

### BACKGROUND OF THE INVENTION

Conventional treatment of cancer with anticancer drugs is usually done with oral or parenteral administration of solutions or dispersions of the active drug substance mostly in aqueous vehicle. However, upon administration of such anticancer drug formulations the active drug quickly distribute all over the body, a minor fraction reaching the pathological tumor site while the majority reaches healthy organs where it can cause toxicity.

Many ways have been proposed and investigated to improve the local antitumor activity and reduce the systemic toxicity of anticancer drugs. One such strategy is to design specific prodrugs that can only be activated by enzymes present on the target site while the prodrug remains inactive when residing in healthy non-target tissues. A particular example of such strategy is derivatization of the drug with glucuronic acid as this perfectly chimes in with the body's own glucuronidation mechanism to inactivate drugs and subsequently quickly eliminate them from the body. While this strategy reduces the potential systemic toxicity of the drug, at the tumor site the activity is retained as many tumors show selective upregulation of glucuronidase enzymes that quickly convert the glucuronide prodrug back into the active parent drug. This way glucuronidation can be seen as a clever strategy to achieve a selectively enhanced effect of an anticancer drug at the target site while reducing its systemic activity and toxicity.

However, the problem of low efficiency of tumor delivery with conventional anticancer drug formulations is not solved with glucuronidation. Indeed, advanced drug delivery systems are needed to improve tumor-specific drug accumulation, a technique commonly referred to as tumor-targeted drug delivery. Of the ones that have been proposed and investigated for this purpose the majority are nanoparticulate formulations, i.e. they are based on particles in the sub-micrometer range, which are engineered to optimize the entrapment and delivery of drugs as well as to ensure biocompatibility and safety. Successful nanoparticulate formulations for drug delivery include liposomes, lipid nanoparticles, polymeric nanoparticles, polymeric micelles and plasma protein-based formulations. Some of the nanoparticulate drug formulations have successfully been evaluated in clinical studies and developed into anticancer nanomedicine products. Manufacturers of these products usually claim improved safety, pharmacokinetics or better efficacy in well-defined treatment regimens and combination therapies.

To achieve sufficient tumor-targeted delivery and avoid premature release in the circulation specific engineering and optimization of the nanoparticle physicochemical properties, morphology and composition is paramount. One such feature is the size of the nanoparticle with very small (i.e. around 50 nm diameter or smaller) being preferred over the more classical liposome and polymeric nanoparticle size of around 100 nm, as very small nanoparticles show a better ability to penetrate deep into the tumors after local extravasation. Nanoparticles of around 50 nm or smaller and still suitable for drug delivery are difficult to formulate, and so far only polymeric particles or polymeric micelles have shown real promise. Especially sufficient drug retention is a critical issue at this size range.

To improve drug encapsulation and retention the anticancer drugs can be chemically optimized and derivatized so as to increase their binding to the nanoparticulate delivery system. An obvious strategy is to chemically connect the drug molecule with the polymers used as excipients and this strategy has successfully been employed, albeit with the drawback that polymers often offer limited capacity for drug coupling as this significantly changes their ability to form nanoparticles. Another disadvantage of chemical connection to the nanoparticle-forming excipients is the criticality of the reversibility of this connection at the target site, which is needed to make the drug fully available for pharmacologic action. From this perspective a better strategy may be to enhance drug encapsulation in and retention by the nanoparticle by chemical derivatization thereby increasing the hydrophobic interaction of the anticancer drug with the excipients that form the hydrophobic interior of the nanoparticles. This strategy has extensively been explored in the prior art.

Chemical derivatization with glucuronic acid, which would impart target selectivity as described above, as such would render the drug too hydrophilic to be incorporated in the hydrophobic interior of the nanoparticles. An aromatic spacer molecule is described in the art that enables quick conversion of the glucuronide prodrug into the active parent drug at the tumor site. Such spacer molecule could lead to a change in physicochemical properties of the prodrug, potentially making it more hydrophobic and thereby enabling better drug encapsulation in the hydrophobic interior of the micelles.

Leenders et al. 1999 and Houba et al. 1998 describe a glucuronic acid prodrug form of doxorubicin (DOX-GA) or daunorubicin (daunorubicin-GA3) employing such spacer molecule and in order to decrease toxicity, which were effective in treatment of different types of tumors in mice.

De Graaf et al. 2004 describes a methylester glucuronidation of doxorubicin (glucuronide prodrug DOX-mGA3) employing a spacer molecule and state that an advantage over DOX-GA3 is that it increases lipophilicity of the prodrug thus having improved pharmacokinetic properties. In addition, release of the active compound is slower because the methyl glucuronate prodrug will be hydrolyzed first to liberate its glucuronide by esterases. The formed acid is further cleaved by glucuronidase and spontaneous release of CO₂ to the drug-spacer molecule. The electron-releasing free amino-group will trigger the 1,6-elimination process and second molecule of CO₂ resulting in release of the parent anthracycline and iminoquinone methide.

EP2098533 describes glucuronide prodrugs of anthracyclines containing one spacer molecule by means of which glucuronic acid is attached to the active compound. EP2098534 describes esters of the glucuronide prodrug containing one or two spacer molecules but not the unesterified glucuronic acid moiety.

However, the doxorubicin glucuronide prodrug made with the spacer molecule as described in the art reported above doesn't load well into micelles. The loading takes a lot of effort and the resulting particles show poor drug retention. Thus far stable doxorubicin glucuronic acid prodrug containing polymeric micelles could only be prepared by covalently attaching the prodrug the polymeric micelle (Talleli et al. 2011 and Ruiz-Hernandez et al. 2014). This has the disadvantage that multi-step reactions are needed to liberate the active compound at the tumor site resulting in a delay in treatment.

Hence, there remains a need in the art to provide improved drug targeting of hydrophilic drug molecules, such as anticancer, antifungal and antibiotic agents.

### SUMMARY OF THE INVENTION

It is an object of the present invention to provide an improved polymeric micelle based drug treatment, in particular wherein the drug is noncovalently bound and incorporated in the hydrophobic interior of nanoparticles in a way that a uniform small nanomedicine is formed that sufficiently loads and retains the agent to achieve targeted delivery for improved therapeutic benefit. The invention therefore provides a polymeric micelle comprising a block copolymer comprising a polyethylene glycol (PEG) hydrophilic block and a hydrophobic block, and a compound according to formula (I) encapsulated within said polymeric micelle

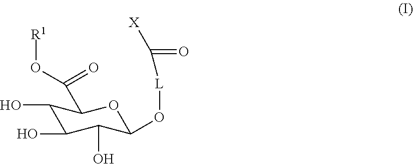

wherein
X is a hydrophilic drug molecule,
R¹ is H or —(CH₂)ₚ—CH₃, wherein p is 0, 1, 2 or 3, or R¹ is a drug molecule and
L is a spacer molecule comprising
   2 to 6 aromatic rings oriented in a linear manner, or
   1-5 aromatic rings oriented in a linear manner and one or more aromatic rings as pendant side groups or
   1-5 aromatic rings and one or more double carbon-carbon bonds oriented in a linear manner,
wherein said rings are each optionally and independently substituted with at least one halogen atom, hydroxyl or alkoxy group, and/or at least one (C1-4)-alkyl.

In another aspect, the invention provides a polymeric micelle comprising a block copolymer comprising a polyethylene glycol (PEG) hydrophilic block and a hydrophobic block, and a compound according to formula (III) encapsulated within said polymeric micelle

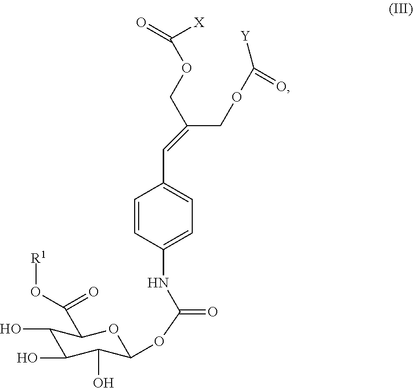

wherein
X and Y are independently a hydrophilic drug molecule, and
R¹ is H or —(CH₂)ₚ—CH₃, wherein p is 0, 1, 2 or 3, or R′ is a drug molecule.

In a preferred embodiment the compound is a compound according to formula (I).

In a further preferred embodiment, the compound is a compound according to formula (I) and R¹ is H or —(CH₂)ₚ—CH₃, wherein p is 0, 1, 2 or 3, more preferably H. In preferred embodiments, L is a spacer molecule comprising 2 to 6 aromatic rings, wherein said rings are each optionally and independently substituted with at least one halogen atom and/or at least one (C1-4)-alkyl.

In a further aspect, the invention provides a pharmaceutical composition comprising a polymeric micelle according to the invention and at least one pharmaceutically acceptable carrier, diluent or excipient.

In a further aspect, the invention provides a polymeric micelle according to the invention for use in therapy.

In a further aspect, the invention provides a method for the treatment or prevention of cancer or infection in a subject in need thereof, comprising administering to the subject a therapeutically effective amount of a polymeric micelle according to the invention.

In a further aspect, the invention provides a polymeric micelle according to the invention for use in a method for the treatment or prevention of cancer or infection.

In a further aspect, the invention provides a use of a polymeric micelle according to the invention in the manufacture of a medicament for the treatment or prevention of cancer or infection.

In a further aspect, the invention provides a method of immunotherapy in a subject in need thereof, the method comprising administering to the subject, preferably a subject suffering from cancer, a therapeutically effective amount of a polymeric micelle according to the invention.

In a further aspect, the invention provides a polymeric micelle according to the invention for use in a method of immunotherapy, preferably in a subject suffering from cancer or infection.

In a further aspect, the invention provides a use of a polymeric micelle according to the invention in the manufacture of a medicament for immunotherapy, preferably in a subject suffering from cancer.

In a further aspect, the invention provides a method for enhancing efficacy of immunotherapy, preferably antibody-based immunotherapy, in a subject suffering from cancer and being treated with said immunotherapy, the method comprising administering to the subject a therapeutically effective amount of a polymeric micelle according to the invention.

In a further aspect, the invention provides a polymeric micelle according to the invention for use in a method for enhancing efficacy of immunotherapy, preferably antibody-based immunotherapy in a subject suffering from cancer and being treated with said immunotherapy.

In a further aspect, the invention provides a use of a polymeric micelle according to the invention in the manufacture of a medicament for enhancing efficacy of immunotherapy, preferably antibody-based immunotherapy, in a subject suffering from cancer and being treated with said immunotherapy.

In a further aspect, the invention provides a use of a polymeric micelle according to the invention in the manufacture of a medicament for enhancing efficacy of immunotherapy, preferably antibody-based immunotherapy, in a subject suffering from cancer and being treated with said immunotherapy.

### DETAILED DESCRIPTION

The present invention effectively combines the benefits of glucuronidation with the benefits of nanoparticulate targeted drug delivery in terms of conferring improved target selectivity to the drug. The invention allows a hydrophilic glucuronide prodrug to show sufficiently strong affinity for the interior of the nanoparticles for optimal drug retention in the particle but also to show sufficient release and conversion of the prodrug into active parent drug at the target site. The invention is based on the finding that glucuronide prodrugs of therapeutically active agents using a spacer comprising 2 to 6 aromatic rings, preferably aryl rings, or using a spacer comprising 1 to 5 aromatic rings, preferably aryl rings, with one or more extra double bonds in the linear sequence, or one or more aromatic rings, preferably aryl rings as pendant side groups show surprising strong affinity for small micellar drug delivery vehicles, the micelles having a hydrophobic core and a hydrophilic shell. Specifically, it was found that if instead of a spacer containing one single aromatic group the spacer has 2 or more aromatic groups in between glucuronide and drug molecule, unexpectedly the physicochemical properties of the prodrug molecule improve, enabling quick formation of well-defined, small and stable polymeric micelles with a relatively high drug loading efficiency.

Glucuronide prodrugs of hydrophilic drug molecules without such spacers are unsuitable for non-covalent encapsulation by polymeric micelles because these are not sufficiently hydrophobic for polymeric micelles having a hydrophobic core. Also glucuronides coupled to hydrophilic drug molecules with a spacer containing one aromatic group are less suitable for encapsulation. Micelles can be made with these prodrugs but the process of drug solubilization and micelle formation is tedious, leads to a less well-defined micelle size range and does not lead to sufficient drug retention for targeted delivery. The results obtained by the present inventors are surprising because, while adding only one extra aromatic group to a single spacer prodrug is only expected to slightly change the log P and the hydrophobicity of the overall chemical structure of the prodrug, the present inventors have now successfully demonstrated that the use of a spacer comprising at least two phenyl groups, at least one phenyl group and one double bond in the linear sequence or at least one phenyl ring and one or more aromatic rings, preferably a phenyl ring, as pendant side groups, or at least two spacers with one phenyl group leads to satisfactory non-covalent encapsulation of the drug in the micelles with the right size and size distribution properties, sufficient loading capacity and drug retention. As demonstrated in the examples, there is a clear effect of adding the double spacer glucuronide prodrug to the empty micelle mixture, which leads to the formation of a small (close to 50 nm diameter) drug-loaded micelle.

The invention therefore provides a polymeric micelle comprising a block copolymer comprising a polyethylene glycol (PEG) hydrophilic block and a hydrophobic block, and a compound according to formula (I) or formula (III) encapsulated within said polymeric micelle

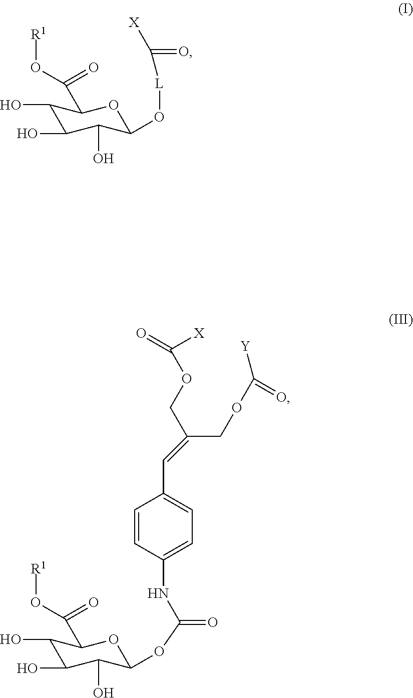

wherein
X and Y are independently a hydrophilic drug molecule,
R¹ is H or —(CH₂)ₚ—CH₃, wherein p is 0, 1, 2 or 3, or R¹ is a drug molecule and
L is a spacer molecule comprising:
   2 to 6 aromatic rings oriented in a linear manner, or
   1-5 aromatic rings oriented in a linear manner and one or more aromatic rings as pendant side groups or
   1-5 aromatic rings and one or more double carbon-carbon bonds oriented in a linear manner,
wherein said rings are each optionally and independently substituted with at least one halogen atom, hydroxyl or alkoxy group, and/or at least one (C1-4)-alkyl

As used herein the term “copolymer” means a polymer wherein at least two types of monomers are present.

“Amphiphilic” means possessing both hydrophobic and hydrophilic properties.

“Hydrophobic” means having a partition coefficient log P in octanol-water higher than 1. For the definition of log P reference is made to Chemical Reviews 1971, volume 71, number 6.

“Hydrophilic” means having a partition coefficient log P in octanol-water lower than 2.

“Drug molecule” as used herein refers to any molecule that has a pharmacological effect when administered to a subject. A preferred drug molecule is an anticancer agent, an antifungal agent, an antibiotic agent or a combination thereof.

“Linker” means a chemical group having functionality to connect different parts (or building blocks) of a molecule, typically two parts. Degradable means in this context biodegradable. “Hydrolysable” means degradable by means of hydrolysis. “Physiological conditions” are the conditions present in an organism to be treated with the controlled release system of the invention; for humans these conditions encompass a pH of about 7.4 and a temperature of about 37° C.

As used herein, the term “subject” encompasses humans and animals, preferably mammals. Preferably, a subject is a mammal, more preferably a human.

The term “therapeutically effective amount,” as used herein, refers to an amount of a compound being administered sufficient to relieve one or more of the symptoms of the disease or condition being treated to some extent. This can be a reduction or alleviation of symptoms, reduction or alleviation of causes of the disease or condition or any other desired therapeutic effect.

As used herein, the term “prevention” refers to preventing or delaying the onset of a disease or condition and/or the appearance of clinical symptoms of the disease or condition in a subject that does not yet experience clinical symptoms of the disease. Prevention of cancer encompasses and in particular refers to recurrence of cancer, for instance following successful treatment of an earlier cancer of the same type. The term “treatment” refers to inhibiting the disease or condition, in particular cancer, i.e. halting or reducing its development or at least one clinical symptom of the disease or condition, and/or to relieving symptoms of the disease or condition.

The polymeric micelle according to the invention comprise a block copolymer comprising a PEG hydrophilic block and a hydrophobic block. The block copolymer is therefore an amphiphilic block copolymer. In aqueous solutions, these polymers form micelles with a hydrophobic core, which can be loaded, particularly with hydrophobic molecules, such as active ingredients. The copolymer of the invention is suitable for use in controlled release systems, target delivery systems and other systems used inside a human or animal body. In some embodiments, this may mean that the copolymer of the invention is a bioresorbable polymer or it converts to a bioresorbable polymer after hydrolysis, which means that the polymer material is safely absorbed by the body and it disappears from the body over time.

Preferably, the polymeric micelle has a hydrophobic core and the hydrophilic anticancer agent in prodrug form is non-covalently entrapped in the hydrophobic core of the micelle.

The polymers of the present invention can have all possible polymer architectures, such as (multi-)block copolymers (such as AB, ABA, ABAB, etc.) or graft copolymers, random copolymers or terpolymers, or a polymeric network; all of which may be grafted.

The hydrophobic block is preferably selected from the group consisting of acrylates, methacrylates, acrylamides, methacrylamides and vinylethers, including lactate and/or aromatic group containing acrylates, methacrylates, acrylamides, methacrylamides and vinylethers. Aromatic group containing acrylates, methacrylates, acrylamides, methacrylamides and vinylethers are most preferred, most preferably methacrylamide or hydroxyalkyl methacrylamide. Examples of preferred monomers include 2-hydroxyethyl methacrylate (HEMA), 2-hydroxyethyl acrylate (HEA), glyceryl methacrylate or glycidyl methacrylate (GMA), glyceryl acrylate or glycidyl acrylate (GA), hydroxypropyl methacrylamide (HPMAAm).

Lactate groups mean a monolactate, a dilactate or an oligolactate group. A lactate can be used to provide the block copolymer with thermosensitive properties. Monolactate groups are preferred.

In a preferred embodiment at least part of the hydrophobic block contains an aromatic side group, such as at least 25 mol. % of the monomers of the hydrophobic block, more preferably at least 50 mol. %. More preferably, at least 75 mol. % of monomers of the hydrophobic blocks contain the aromatic side group and most preferably essentially 100 mol. % or 100 mol. % of monomers of the hydrophobic blocks.

As used herein “Aromatic side group” means that the side group contains at least one aromatic ring, preferably one aromatic ring or a fused bicyclic aromatic ring. A preferred aromatic side group is selected from the group consisting of benzyl, benzoyl, phenyl, naphthyl, biphenyl, and indoyl.

“Aromatic ring” as used herein encompass both aryl rings and heteroaryl rings, preferably an aromatic ring is an aryl ring, i.e. does not contain heteroatoms.

Aryl mas used herein refer to a monocyclic or polycyclic aromatic hydrocarbon group derived from the corresponding arene by removal of a hydrogen atom from a ring carbon atom. As used herein a heteroaryl ring is an aryl ring containing at least one heteroatom, preferably N, O or S, preferably one heteroatom selected from N, O and S.

The aromatic side group is preferably coupled to the polymer of the hydrophobic block via a degradable linker. This ensures a constant release of this group together with the physically entrapped active ingredient. The linker is preferably degradable under physiological conditions, and more preferably it is hydrolysable under physiological conditions. But the linkers might also be hydrolyzed by the actions of enzymes. Generally, known linkers can be used, selected from esters, orthoesters, amides, carbonates, carbamates, anhydrides, ketals, acetals, hydrazone and derivatives thereof. In some embodiments, an ester linker is used, preferably of formula —C(O)—O—. In that case, the phenyl group together with the ester linker forms a benzoyl group, and the naphthyl group together with the ester linker—a naphthoyl group. In yet other embodiments, hydrazone linkers are used. Hydrazone linkers contain a group —C═N—N—.

In a special embodiment of the present invention a pH-sensitive linker is used. Preferably, such linker hydrolyzes at a pH less than 7, more preferably at a pH of 6.5 or less.

An advantage of a pH-sensitive linker is that it can selectively release the entrapped drug at areas with an increased acidity, such as some tumors or inflammation sites. Also degradation of the micelles and release of entrapped drug might occur in endosomes or lysosomes having a lower pH, after cellular binding and internalization of the drug-loaded micelles.

Particularly preferred are hydrazone linkers with the following general formula:

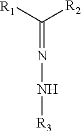

wherein R₁, R₂ can be independently of each other H or alkyl groups, and wherein R₃ is an alkyl group or a ketone group —C(O)—. Preferably, the alkyl group is a C₁-C₃ alkyl. More preferably, R₁ or R₂ is a methyl group. In a preferred embodiment, R₃ is a ketone group —C(O)—.

As polyethylene glycol (PEG) hydrophilic block of the copolymer of the invention any suitable PEG polymer can be used. Examples of suitable PEG polymers are those of a molecular mass between 2000 and 5000 Dalton.

In a particularly preferred embodiment the polymeric micelle according to the invention consist of a block copolymer consisting of the PEG hydrophilic block and the hydrophobic block.

The weight ratio of the hydrophobic to hydrophilic block is preferably in the range from 10:90 to 90:10, more preferably from 40:60 to 60:40.

The polymeric micelles preferably have a size of 20-100 nm. This refers to Z-average hydrodynamic diameter, as measured by dynamic light scattering. The polydispersity index PDI is preferably less than 0.3, more preferably less than 0.15, as measured by dynamic light scattering.

Preferred polymeric micelles are for instance those described in WO 2016/024861, Shi et al. 2013, EP1792927B1 and U.S. Pat. No. 7,820,759B2, which documents are incorporated herein by reference.

The polymeric micelle according to the invention comprises a compound according to formula (I) encapsulated within the micelle

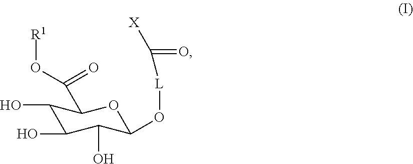

Alternatively or additionally, the polymeric micelle according to the invention comprises a compound according to formula (III) encapsulated within the micelle

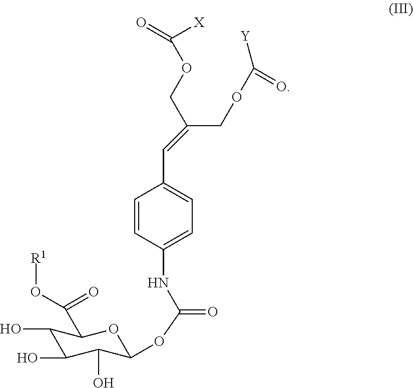

Encapsulation of the compound or active ingredient or ingredients as used herein means that the compound or active ingredient ingredients are not covalently attached to any polymer of the polymeric micelle. “Non-covalent” refers any interaction which is not covalent, i.e. any weak bonding between atoms or bonds which bonding does not involve the sharing of electron pairs. Examples of non-covalent interaction are hydrophobic, aromatic (π-π), hydrogen bonding, electrostatic, stereo complex, and metal-ion interactions. Encapsulation is also used as a synonym for loading or entrapment of the active ingredient.

In formula (I) X is a hydrophilic drug molecule, preferably a hydrophilic anticancer, antifungal or antibiotic agent. In formula (III), both X and Y are independently a hydrophilic drug molecule, preferably a hydrophilic anticancer, antifungal, antibiotic agent or combination thereof. As used herein “independently”, means that the hydrophilic drug for X and the hydrophilic drug for Y may be the same or different. In formula (III), X and Y are preferably two hydrophilic anticancer agents, two hydrophilic antifungal agents or two hydrophilic antibiotic agents. These two agents can be the same or different. In one preferred embodiment X and Y are the same hydrophilic drug molecule in formula (III). In another preferred embodiment, X and Y are different hydrophilic drug molecules in formula (III).

As used herein “hydrophilic” refers to having a log P value of below 2. The hydrophilic drug molecule may comprise a moiety connected that allows for coupling the drug molecule to the spacer molecule, such as an amino acid. As used herein “anticancer agent” refers to a therapeutic agent that is used in the treatment of any type of cancer. As used herein “antifungal agent” refers to a therapeutic agent that is used in the treatment of any type of fungal infection. As used herein “antibiotic agent” refers to a therapeutic agent that is used in the treatment of any type of bacterial infection. Hydrophilic anticancer, antifungal and antibiotic agents are well known in the art and can easily be identified by a skilled person.

The hydrophilic anticancer agent or agents are preferably selected from the group consisting of anthracyclines, of which doxorubicin, daunorubicin, epirubicin and idarubicin are preferred; nucleoside/deoxycytidine analogues, of which gemcitabine, cytarabine, lamivudine, zalcitabine are preferred and their amine-, hydroxyl- and thiol-functionalized analogues and derivatives; topoisomerase I inhibitors, of which irinotecan, topotecan, camptothecin and their amine functionalized analogues are preferred; nitrogen mustard alkylating agents, of which chlorambucil and melphalan are preferred; mitomycin C; immunomodulators (adjuvants), of which toll-like receptor agonists such as resiquimod, imiquimod and gardiquimod, and their amine functionalized analogues such as 1-(4-(aminomethyl)benzyl)-2-butyl-1H-imidazo[4,5-c]quinolin-4-amine are preferred; P-glycoprotein drug efflux pump inhibitors, of which laniquidar, mitotane, biricodar, elacridar and tariquidar and their amine-, hydroxyl- and thiol-functionalized analogues and derivatives are preferred; taxanes, of which paclitaxel and docetaxel and their amine-, hydroxyl and thiol-functionalized analogues and derivatives are preferred; anticancer peptide drug molecules and anticancer nucleic acid compounds; and platinum compounds, of which cisplatin, carboplatin and oxaliplatin and their amine-, hydroxyl- and thiol-functionalized analogues are preferred; These platinum compounds are more preferably cisplatin, oxaliplatin, carboplatin in a dihydroxo-related platinum(IV) prodrug form and can be modified with a succinate or an alpha or beta amino acid linker in one or both axial positions, meaning for example trans-[Pt(NH₃)₂Cl₂(succinato)(OR²)], [Pt(oxalato)(1,2-cyclohexanediamine)(succinato)(OR²)] and [Pt(NH₃)₂(1,1-cyclobutanedicarboxylato)(succinato)(OR²)], respectively. In a preferred embodiment, the anticancer agent is an anthracycline, more preferably doxorubicin, daunorubicin, epirubicin or idarubicin. In another preferred embodiment, the anticancer agent is a nucleoside analogue, more preferably gemcitabine and cytarabine. In another specific preferred embodiment, the anticancer drug is a mustard analogue, preferred molecular formula of a compound of formula (I) wherein the hydrophilic drug molecule is a mustard analogue are shown below:

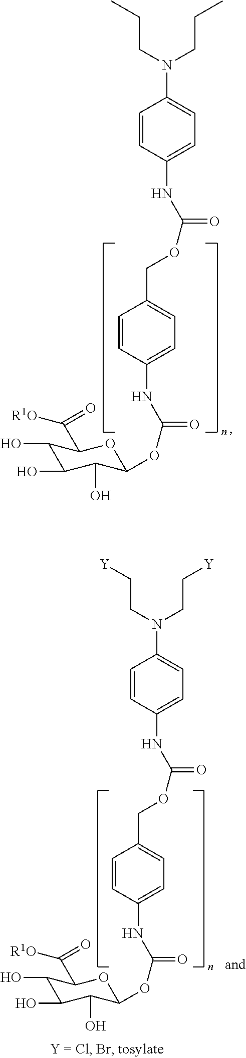

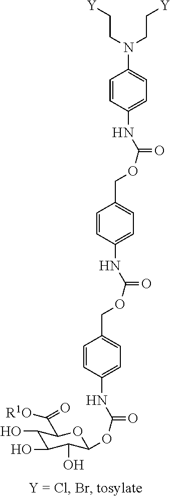

The hydrophilic antifungal agent or agents are preferably selected from the group consisting of amphotericin B, candicidin, hamycin, nystatin and rimocidin.

The hydrophilic antibiotic agent or agents are preferably selected from the group consisting of amikacin, gentamicin, kanamycin, neomycin, streptomycin and tobramycin.

In a preferred embodiment, R¹ is H or CH₃ in formula (I) or formula (III). In a particularly preferred embodiment, R¹ is H in formula (I) and, hence, a polymeric micelle according to the invention comprises a compound according to formula (II) encapsulated within the micelle

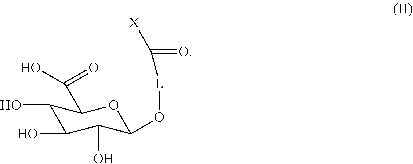

In another preferred embodiment R¹ is a drug molecule in formula (I) or formula (III). Preferred drug molecules are platinum compounds. The platinum compound is preferably cisplatin, oxaliplatin or carboplatin. The platinum compounds can be coupled using the methyl ester of the glucuronic moiety, after deprotection with LiOMe. The coordination of the —COOH group of the glucuronic moiety to the related Pt(IV) prodrug form of cisplatin, oxaliplatin, carboplatin or related goes via the trans-hydroxo group of platinum, mediated by the use of activating agents such as DCC or HATU.

Particularly preferred are compounds of the following formulas:

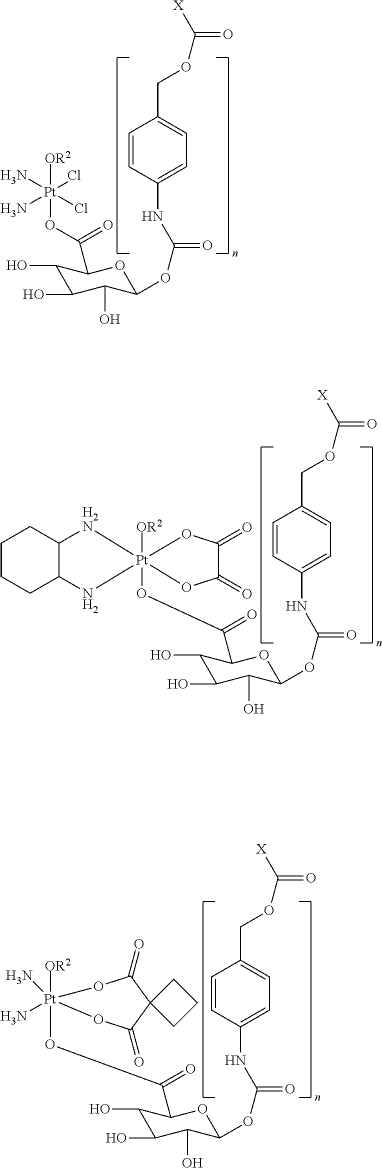

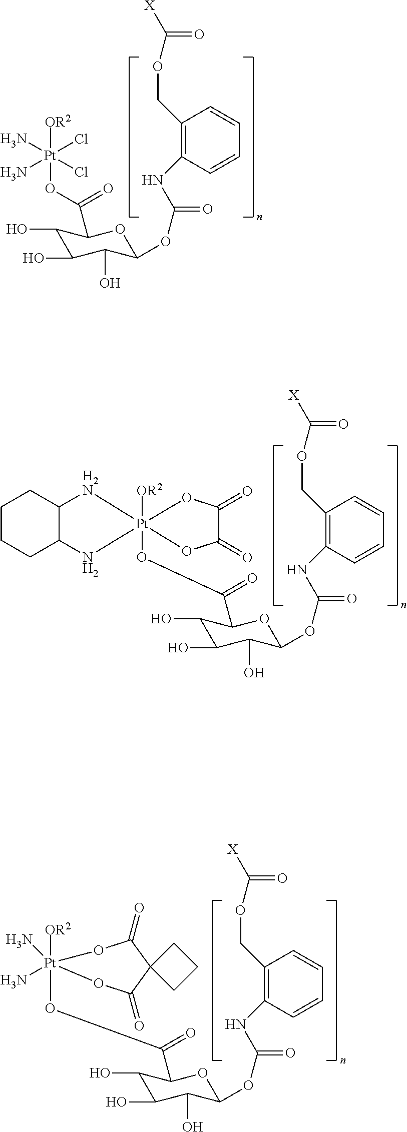

In formula (I), L is a spacer molecule comprising 1 to 6 aromatic rings.

In one embodiment the spacer molecule comprises 2 to 6 aromatic rings, whereby said rings are orientated in a linear manner, meaning that none of the aromatic rings is a substituent of another aromatic ring, but rather form a chain of aromatic rings. Optionally, the chain may comprise other atoms, such as one or more carbon atoms and/or one or more oxygen atoms between aromatic rings.

In another embodiment the spacer molecule comprises 1 to 5 aromatic rings oriented in a linear manner and one or more aromatic rings as pendant groups. “Oriented in a linear manner” as used herein means that none of the aromatic rings is a substituent of another aromatic ring, but rather form a chain of aromatic rings. Optionally, the chain may comprise other atoms, such as one or more carbon atoms and/or one or more oxygen atoms between aromatic rings. As used herein “pendant group” means that the aromatic ring or aromatic rings are attached to the backbone that is formed by the chain of one or more aromatic rings and optionally further atoms, such as one or more carbon atoms and/or one or more oxygen atoms.

In a preferred embodiment, the spacer molecule comprises 1 to 5 aromatic rings oriented in a linear manner and 1 to 3 aromatic rings as pendant side group, more preferably 1 or 2 aromatic rings as pendant side groups, more preferably 1 aromatic ring as a pendant side group. Such aromatic ring as a pendant side group is for instance obtained when a drug molecule coupled to an amino acid containing an aromatic ring (such as phenylalanine) is attached to the spacer molecule via the amine group of the amino acid.

In another embodiment the spacer molecule comprises 1 to 5 aromatic rings and one or more double (unsaturated) carbon-carbon bonds oriented in a linear manner. I.e. the one or more double carbon-carbon bonds are present in an aliphatic part of the spacer molecule. “Oriented in a linear manner” as used herein means that none of the aromatic rings is a substituent of another aromatic ring, but rather form a chain of aromatic rings. Optionally, the chain may comprises other atoms, such as one or more carbon atoms and/or one or more oxygen atoms between aromatic ring(s) and/or double carbon-carbon bond(s).

Compounds of formula (III), comprise a spacer molecule comprising a bifurcated structure wherein two aliphatic structures are connected to a chain comprising an aromatic ring and a double carbon-carbon bond at one end, and are connected to two hydrophilic drug molecules at the other ends.

Preferably the spacer molecule L in formula (I) contains at most 48 atoms in the combined linear sequences that constituted L. The longest linear atomic sequence between the oxygen atom of the ester group connecting the glucuronic acid to the spacer molecule and C═O moiety connecting the spacer molecule to the hydrophilic drug molecule, preferably anticancer agent, including part of the atoms of the aromatic rings that are part of this sequence, is preferably 8-48 atoms, more preferably 10-48 atoms. For instance, formula (Id) has 10 atoms in the longest linear atomic sequence, formula (Ia) wherein n=6 has 48 atoms in the longest linear atomic sequence and formula (Ib) wherein n=2 has 12 atoms in the longest linear atomic sequence. This longest linear atomic sequence is preferably 10-40 atoms, more preferably 10-32 atoms, more preferably 10-24 atoms, more preferably 10-19 atoms.

The 2 to 6 aromatic rings are optionally each independently substituted with at least one halogen atom, preferably chlorine, and/or at least one (C1-4)-alkyl, preferably methyl or ethyl, more preferably methyl. It is further preferred that each ring contains no, one or two of the optional substituents, preferably no or no optional substituent. In a particularly preferred embodiment, the aromatic rings are unsubstituted.

The aromatic rings are as defined herein above. Preferably the aromatic rings are aryl rings. It is further preferred that the aryl rings are each independently selected from phenyl, naphthyl, biphenyl and combinations thereof.

In a preferred embodiment, the spacer molecule comprises 2 to 5 aromatic rings, preferably aryl rings, more preferably 2 to 4 aromatic rings, preferably aryl rings. The aromatic rings may be combined in any suitable manner, for instance, the spacer molecule may comprise 2 to 6 aromatic rings each connected. In a particularly preferred embodiment, the spacer molecule comprises 2 aryl rings, such as 2 phenyl rings or one naphthyl moiety. In another preferred embodiment, the spacer molecule comprises 3 aryl rings, such as 3 phenyl rings or a combination of a naphthyl moiety and a phenyl ring.

Preferred compounds encapsulated within the polymeric micelle are selected from the group consisting of:

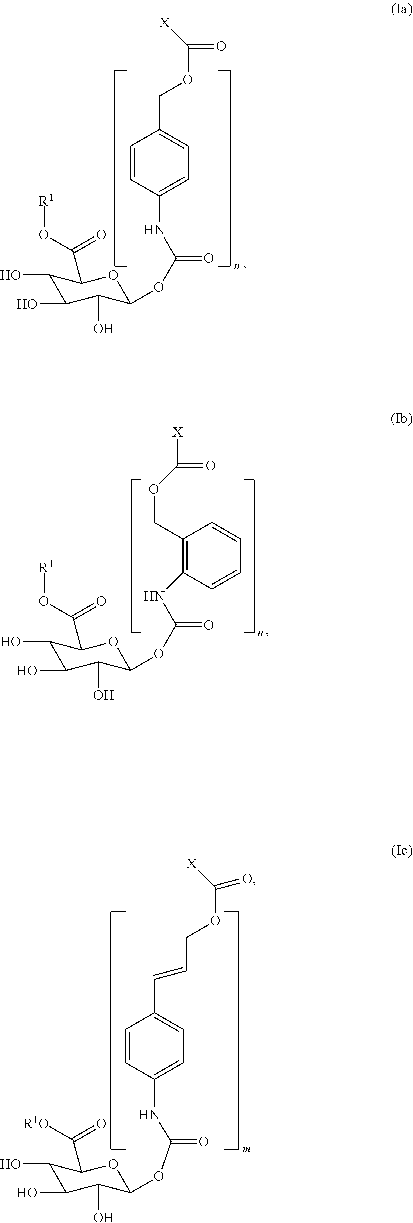

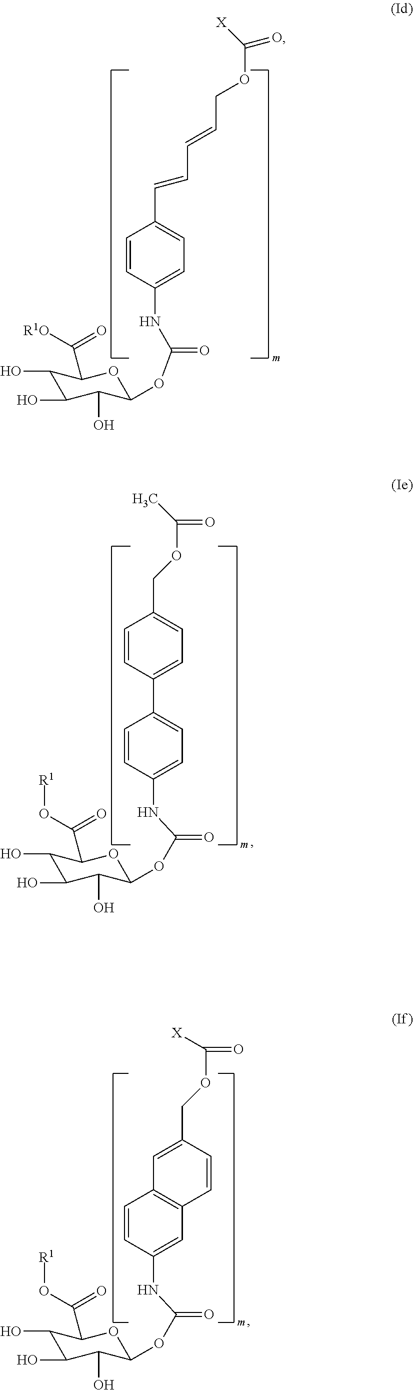

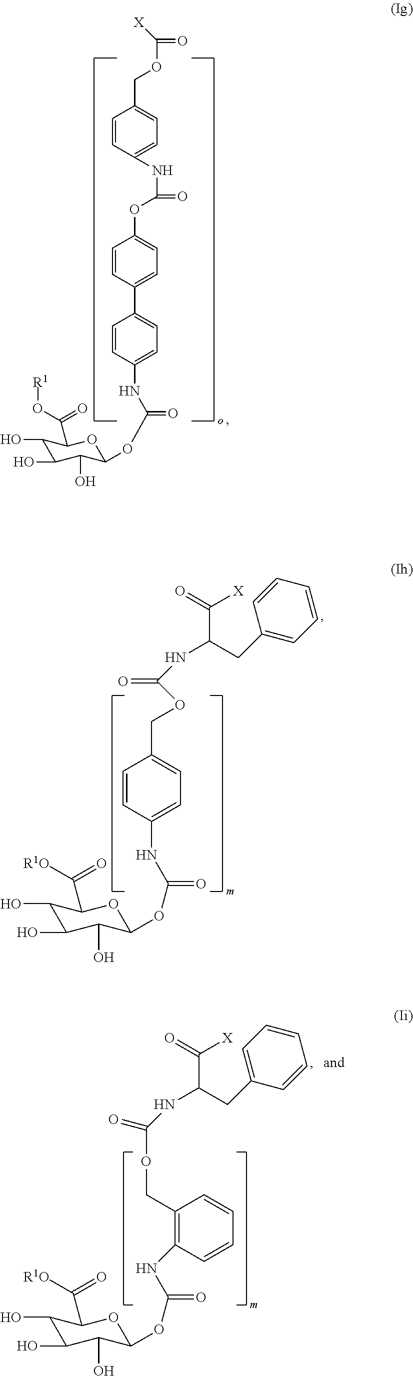

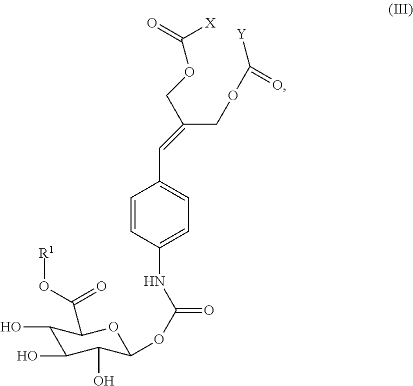

wherein R¹ is H or —(CH₂)ₚ—CH₃, wherein p is 0, 1, 2 or 3, or R¹ is a drug molecule and
wherein n is 2 to 6, m is 1 to 3, and o is 1 or 2.

Preferably R¹ is H of CH₃, more preferably H.

Preferably in formula's (Ia) and (Ib) n is 2 to 5, more preferably 2 to 4.

Preferably in formula's (Ic), (Id) and (Ie) m is 1 or 2, most preferably 1.

Preferably in formula (If) o is 1.

Preferably in formula (Ih) and (Ii) m is 1 or 2, most preferably 1.

A particularly preferred compound is a compound of formula (Ia or b) wherein n is 2 to 5, more preferably 2 or 3, most preferably 2.

Another particularly preferred compound is a compound of formula (Ic or d) wherein m is 1 or 2, most preferably 1.

Another particularly preferred compound is a compound of formula (Ie) wherein m is 1 or 2, most preferably 1.

Another particularly preferred compound is a compound of formula (If) wherein m is 1 or 2, most preferably 1.

Another particularly preferred compound is a compound of formula (Ig), wherein o is 1.

Formula (Ic) is preferably formula (Ic′) and formula (Id) is preferably formula (Id′):

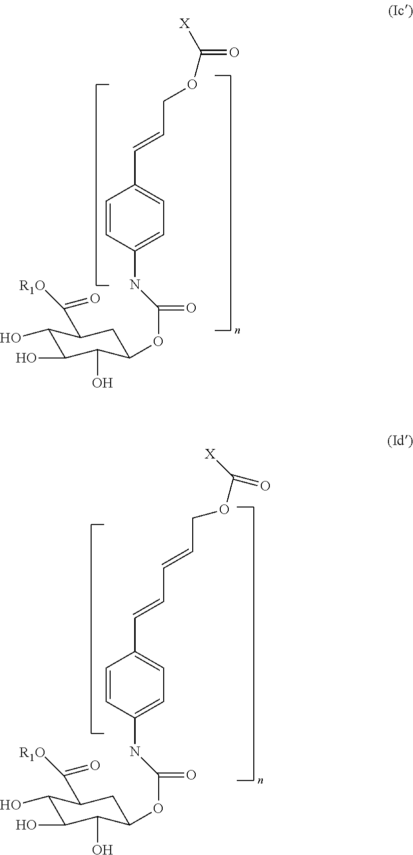

wherein R¹ is H or —(CH₂)ₚ—CH₃, wherein p is 0, 1, 2 or 3, or R¹ is a drug molecule and wherein n is 2 or 3, more preferably n is 2.

In further preferred embodiments, the compound is a compound according to formula (I)

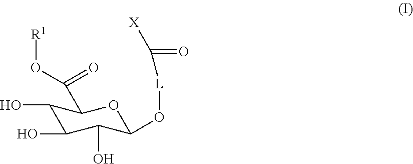

wherein
X is a hydrophilic drug molecule,
R¹ is H or —(CH₂)ₚ—CH₃, wherein p is 0, 1, 2 or 3, preferably R¹ is H, and
L is a spacer molecule comprising 2 to 6 aromatic rings, wherein said rings are each optionally and independently substituted with at least one halogen atom and/or at least one (C1-4)-alkyl.

Provided is therefore a pharmaceutical composition comprising a polymeric micelle according to the invention, and at least one pharmaceutically acceptable carrier, diluent and/or excipient. By “pharmaceutically acceptable” it is meant that the carrier, diluent or excipient must be compatible with the other ingredients of the formulation and not deleterious to the recipient thereof. In general, any pharmaceutically suitable additive which does not interfere with the function of the active compounds can be used. A pharmaceutical composition according to the invention is preferably suitable for human use. A pharmaceutical composition according to the invention is preferably suitable for intravenous administration. Compositions for intravenous administration may for example be solutions of the compounds of the invention in sterile isotonic aqueous buffer. Where necessary, the intravenous compositions may include for instance solubilizing agents, stabilizing agents and/or a local anesthetic to ease the pain at the site of the injection.

A pharmaceutical composition according to the invention preferably comprises a plurality of micelles of the invention. In one embodiment, each of the plurality of micelles is identical comprising the same compound or compounds and the same drug molecule or drug molecules, i.e. all micelles are the same. In other embodiments, the plurality of micelles are not all identical, but may comprise two or more different micelles, for instance micelles comprising different compounds and/or different drug molecules.

Suitable methods for administration of polymeric micelle or a pharmaceutical composition according to the invention can be readily determined by a person skilled in the art. Preferably the polymeric micelles and pharmaceutical compositions are administered intravenously.

The polymeric micelle of the invention is particularly suitable as controlled and/or targeted drug delivery. Targeting is preferably to a site of a tumor.

Also provided is a polymeric micelle according to the invention for use in therapy.

Also provided is a polymeric micelle according to the invention for use in a method for the treatment or prevention of cancer and infections. Also provided is a method for the treatment or prevention of cancer or infections in a subject in need thereof, comprising administering to the subject a therapeutically effective amount of a polymeric micelle according to the invention.

Cancer that can be treated using polymeric micelles according to the invention can be any type of cancer, including primary tumors, secondary tumors, advanced tumors and metastases. A skilled person is able to select appropriate hydrophilic anti-cancer agents for each type of cancer. Non-limiting examples of cancers that can be treated or prevented in accordance with the invention are acute myeloid leukemia (AML), chronic myeloid leukemia (CML), chronic lymphocytic leukemia (CLL), acute lymphoblastic leukemia (ALL), chronic myelomonocytic leukemia (CMML), lymphoma, multiple myeloma, eosinophilic leukemia, hairy cell leukemia, Hodgkin lymphoma, non-Hodgkin lymphoma, large cell immunoblastic lymphoma, plasmacytoma, lung tumors, small cell lung carcinoma, non-small cell lung carcinoma, pancreatic tumors, breast tumors, liver tumors, brain tumors, skin tumors, bone tumors, colon tumors, rectal tumors, anal tumors, tumors of the small intestine, stomach tumors, gliomas, endocrine system tumors, thyroid tumors, esophageal tumors, gastric tumors, uterine tumors, urinary tract tumors and urinary bladder tumors, kidney tumors, renal cell carcinoma, prostate tumors, gall bladder tumors, tumors of the head or neck, ovarian tumors, cervical tumors, glioblastoma, melanoma, chondrosarcoma, fibrosarcoma, endometrial, esophageal, eye or gastrointestinal stromal tumors, liposarcoma, nasopharyngeal, thyroid, vaginal and vulvar tumors.

A micelle according to the invention is suitably combined with one or more other drug molecules.

Further, different micelles according to the invention comprising different drug molecules are suitably combined into a pharmaceutical composition according to the invention.

Further, multiple compounds and/or multiple drug molecules are suitably combined into one micelle according to the invention.

In one exemplary embodiment, a micelle is loaded with multiple compounds of formula (I) and/or formula (III), wherein in each compound of formula (I) and/or each compound of formula (III) the hydrophilic drug molecule X and optionally Y is a different drug molecule. For instance, two anticancer agents are combined in a micelle according to the invention, such as doxorubicin and tariquidar. As another example, an anticancer agent and an antibiotic or antifungal agent are combined in a micelle according to the invention.

In another exemplary embodiment, a pharmaceutical composition of the invention comprises multiple micelles each loaded with one type of compound of formula (I) or formula (III), wherein in each compound of formula (III) the hydrophilic drug molecule X and optionally Y is a different drug molecule.

An hydrophilic anticancer, antifungal or antibiotic agent may also be combined with yet another drug molecule, encapsulated within the micelle or not, such as doxorubicin and losartan.

The polymeric micelles wherein the hydrophilic drug molecule is a hydrophilic anticancer agent according to the invention are also advantageously combined with immunotherapy, in particular tumor immunotherapy. Cancer immunotherapy suffers from the limitation that the majority of patients do not respond to immunotherapeutics such as checkpoint blockade antibodies. These patients with non-responsive tumors (“cold tumors”) can be primed by other treatments to make their tumors responsive to immunotherapy (“hot tumors”). One important strategy to do this is by chemotherapeutic drugs that are able to induce immunogenic cell death, which is a specific type of cell death that is highly immunogenic and can activate the immune system. Decades of research has shown that cancer immunotherapy works more efficiently with activated immune systems in cancer patients. However, chemotherapy also has immunosuppressive side effects such as the depletion of lymphocytes. It has been shown that by loading them into nanoparticles the immunosuppressive side effects of immunogenic chemotherapeutics can be minimized, which resulted in more efficient immunoactivation (ref: Rios-Doria, J.; Durham, N.; Wetzel, L.; Rothstein, R.; Chesebrough, J.; Holoweckyj, N.; Zhao, W.; Leow, C. C.; Hollingsworth, R. Doxil synergizes with cancer immunotherapies to enhance antitumor responses in syngeneic mouse models. Neoplasia. 2015, 17, 661-670; Zhao, X.; Yang, K.; Zhao, R.; Ji, T.; Wang, X.; Yang, X.; Zhang, Y; Cheng, K.; Liu, S.; Hao, J.; Ren, H.; Leong, K. W.; Nie, G. Inducing enhanced immunogenic cell death with nanocarrier-based drug delivery systems for pancreatic cancer therapy. Biomaterials 2016, 102, 187-197). Loading the e.g. a DOX prodrug, but also other anticancer agents, in micelles may have similar effects on immunoactivation, and this may be combined with antibodies that modulates the immunity, such as the checkpoint blockade inhibitors.

“Immunotherapy” as used herein refers to treatment of an individual suffering from a disease or disorder by inducing or enhancing an immune response in said individual. Tumor immunotherapy relates to inducing or enhancing an individual's immune response against a tumor and/or cells of said tumor Immunotherapy according to the invention can be either for treatment or prevention. Prevention of cancer is preferably prevention of recurrence of cancer, for instance following successful treatment of an earlier cancer of the same type.

Hence, in one embodiment, treatment based on polymeric micelles according to the invention is combined with at least one immunotherapy component. Such immunotherapy component can be any immunotherapy component known in the art. Preferably, said immunotherapy component is selected from the group consisting of cellular immunotherapy, antibody therapy, cytokine therapy, vaccination and/or small molecule immunotherapy, or combinations thereof. In a preferred embodiment, treatment with polymeric micelles according to the invention is combined with antibody-based immunotherapy, preferably comprising treatment using antibodies directed against a co-inhibitory T cell molecule.

Also provided is therefore a polymeric micelle according to the invention for use in a method for enhancing efficacy of immunotherapy, preferably antibody-based immunotherapy in a subject suffering from cancer and being treated with said immunotherapy. Also provided is a method for enhancing efficacy of immunotherapy, in particular antibody-based immunotherapy, in a subject suffering from cancer and being treated with said immunotherapy, the method comprising administering to the subject a therapeutically effective amount of a polymeric micelle according to the invention.

“Enhancing” as used herein preferably means that the efficacy of immunotherapy is increased by at least about 10%, preferably at least about 15%, more preferably at least about 20%, such as about 25%, about 50%, about 75%, about 80%, about 85%, about 90%, or about 95%.

“Efficacy of immunotherapy” in as subject suffering from cancer refers to an inhibitory effect on tumor growth, which effect can be manifested as slowing, retarding, arresting or even reversing growth of a tumor.

Co-inhibitory T cell molecules are also referred to as immune checkpoints. Preferred examples of co-inhibitory T cell molecules are cytotoxic T-lymphocyte antigen-4 (CTLA-4), programmed death-1 (PD-1), PD-ligand 1 (PD-L1), PD-L2, Signal-regulatory protein alpha (SIRPα), T-cell immunoglobulin- and mucin domain-3-containing molecule 3 (TIM3), lymphocyte-activation gene 3 (LAG3), killer cell immunoglobulin-like receptor (KIR), CD276, CD272, A2AR, VISTA and indoleamine 2,3 dioxygenase (IDO). An antibody against a co-inhibitory T cell molecule that is combined with a chimeric receptor or cell comprising a chimeric receptor according to the invention is therefore preferably selected from the group consisting of an anti-CTLA4 antibody, an anti-PD-1 antibody, an anti-PD-L1 antibody, an anti-PD-L2 antibody, an anti-SIRPα antibody, an anti-TIM3 antibody, an anti-LAG3 antibody, an anti-CD276 antibody, an anti-CD272 antibody, an anti-KIR antibody, an anti-A2AR antibody, an anti-VISTA antibody, anti TIGIT antibody and an anti-IDO antibody. Suitable antibodies used as an antibody-based immunotherapy component are nivolumab, pembrolizumab, lambrolizumab, ipilimumab, and lirilumab.

Methods to prepare polymeric micelles and load such micelles with a hydrophilic anticancer agent such that it is non-covalently encapsulated are well known to a person skilled in the art and are described in the examples herein.

The copolymer can for instance be prepared by starting from a mixture of the monomers and carrying out the polymerization reaction. Preferably, the copolymer is obtained by free radical polymerization. It is also possible to first produce the copolymer and subsequently functionalize it by coupling suitable groups. Methods of synthesis of copolymers are known to a skilled person. An example of a suitable method to synthesize monomers is described in Shi et al., 2013. An example of a synthesis of macroinitiator that can be used in the polymerization reaction is described in Neradovic et al. 2001. Further suitable methods are described in WO 2016/024861.

The compound of formula (I) or formula (III) can be prepared by methods known in the art for coupling a glucuronic acid via a spacer molecule to a hydrophilic drug molecule and optionally a further drug molecule at position R¹.

Suitable methods are described in the examples herein and in De Graaf et al. 2004, Houba et al. 1998, Houba et al. 2001, Leenders et al. 1999, De Groot et al. 2001, EP2098534 and Tranoy-Opalinski et al. 2008, which documents are incorporated herein by reference.

Methods for loading the polymeric micelles with a glucuronic acid prodrug form of a hydrophilic anticancer agent as described herein are also known in the art. Suitable methods are described in the examples herein and in Shi et al., 2013 and Bagheri et al, 2018, which documents are incorporated herein by reference.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: 1H-NMR spectrum of DOX GA3-DS.

FIG. 2: HPLC chromatogram of DOX GA3-DS.

FIG. 3: Conversion of DOX prodrugs to DOX in the presence of beta-GUS.

FIG. 4: Transmission electron micrographs of micelles loaded with 5 mg/mL doxorubicin double spacer prodrug (left panel) and single spacer prodrug (right panel).

FIG. 5: Release of DOX and prodrugs from the micelles.

FIG. 6: In vitro calreticulin (CRT) translocation of 4T1 cancer cells treated with DOX, prodrug and DOX prodrug-loaded micelles. 3A (left panel) shows the higher signal in the flow cytometry plots, which in 3B (right panel) is further quantified.

FIG. 7: Experimental setup of the in vivo study

FIG. 8: Quantification of CD3+CD8+ cytotoxic T cells in tumors after treatment assessed by flow cytometry.

FIG. 9: Quantification of CD3+CD8+ cytotoxic T cells in tumors after treatment assessed by fluorescence microscope.

FIG. 10: 1H-NMR spectrum of GEM-Phe-mGA3.

FIG. 11: HPLC chromatogram of GEM-Phe-mGA3.

FIG. 12: Z-average of GEM, GEM-Phe, and GEM-Phe-mGA encapsulated in micelles as measured by dynamic light scattering (DLS).

FIG. 13: Loading capacity (LC) and encapsulation efficiency (EE) of GEM, GEM-Phe, and GEM-Phe-mGA encapsulated in micelles.

FIG. 14: Release of GEM, GEM-Phe, and GEM-Phe-mGA from the micelles over time as assessed by the dialysis method.

### EXAMPLES

### Example 1. Synthesis and Micelle Loading of a DOX-Glucuronic Acid Double Spacer Prodrug

Prodrug Synthesis

The synthesis of the prodrug DOX-GA3 double spacer was divided into 3 parts. Detailed information is shown below.

Part 1: Synthesis of the Acetyl-Protected Methyl Ester of Glucuronic Acid

Firstly, methyl 1,2,3,4-tetra-O-acetyl-6-D-glucopyranuronate (3) was prepared by pyridine-catalyzed acetylation from the starting commercially available D-(+)-Glucuronic acid γ-lactone (1) according to Bollenback et al in: The Synthesis of Aryl-D-glucopyranosiduronic Acids, 1954, pp. 3310-3315. And compound (4) with a free anomeric hydroxyl was prepared through catalyzing compound (3) with ammonium carbonate associated with potassium bisulfate according to Bosco et al in: Lewis-Acidic Polyoxometalates as Reusable Catalysts for the Synthesis of Glucuronic Acid Esters under Microwave Irradiation, 2010, pp. 1249-1252, and purified by column chromatography. As show in scheme 1.

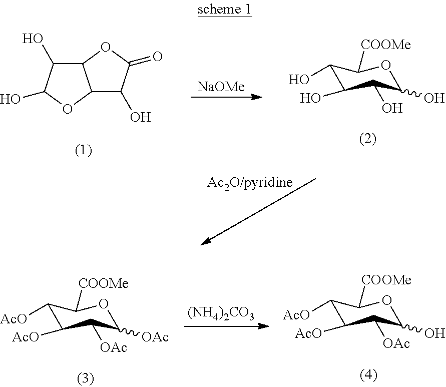

Part 2: Synthesis of p-t.Butyldimethylsilyloxymethyl Benzoic Acid

Saponification of the esters from the commercially available compound (5) to carboxylic acids (6), followed by protection using tert-butyldimethylsilyl chloride (TBDMSCl) to yield silyl ethers (8). The specific procedures are shown in scheme 2.

From (5) to (6). 60 mmol methyl-(p-hydroxymethyl) benzoate (5) and 120 mmol KOH were dissolved in MeOH/water (3/1) mixture. After 2 h of refluxing, MeOH was evaporated and 1M HCl was added. The compound (6) formed crystals and were collected by filtration.

From (6) to (8). 13.1 mmol of (6), 39.7 mmol of imidazole and 33.2 mmol of t.butyldimethylsilyl chloride were dissolved in dry THF. After overnight reaction under nitrogen, saturated NaHCO₃ was added to the THF solution to saponify the silyl ester. After complete hydrolysis, the water layer was extracted with ethyl acetate and washed with saturated NaCl to get the compound (8).

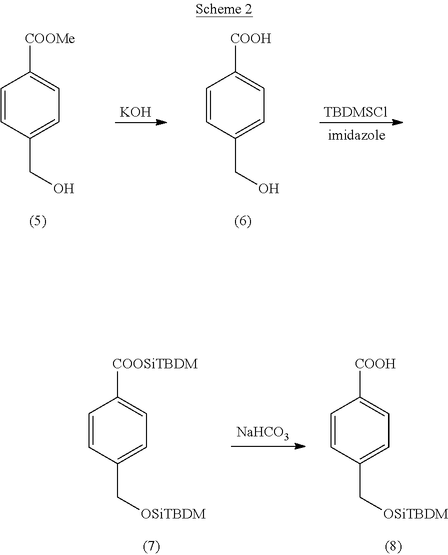

Part 3: Synthesis of the Final DOX-GA3 Double Spacer Prodrug

Synthesis of Silyl Protection of Methyl-Glucuronide Single Spacer Compound B (Scheme 3-i, See the End of the Protocol)

2 g (7.52 mmol) of compound 8, 0.9 g (8.9 mmol) of triethylamine and 2.45 g (8.9 mmol) of diphenylphosphoryl azide (DPPA) were dissolved in 40 ml of dry toluene. The mixture was stirred at 85° C. for 3 h under argon atmosphere. Next, 2 g of compound 4 (6 mmol) in 20 ml of toluene was added to the above mixture and the reaction was kept overnight at room temperature. After evaporation of the solvent, the residue was purified by column chromatography (heptane/ethyl acetate=5/2).

Synthesis of Deprotected Silyl of Methyl-Glucuronide Single Spacer Moiety C (Scheme 3-ii)

7.3 g of compound B is hydrolyzed in a mixture water/THF/acetic acid (1/1/1) for 4 h. Then the mixture is concentrated, the residue was dissolved in dichloromethane and purified by column chromatography (heptane/ethyl acetate=2/3).

Synthesis of Silyl Protection of Methyl-Glucuronide Double Spacer Compound D (Scheme 3-iii)

The procedure was similar with the synthesis of compound B. The carboxylic acid group of compound 8 was converted to isocyanate and then 2 g of compound C (4.1 mmol) and 0.42 g of triethylamine (4.1 mmol) in dry toluene and acetonitrile were added. After stirring overnight, the reaction mixture was dissolved in a minimum amount of chloroform and brought on a silica column (heptane/ethyl acetate=2/1).

Synthesis of Deprotected Silyl of Methyl-Glucuronide Double Spacer Moiety E (Scheme 3-iv)

Compound D (4 mmol) was dissolved in 45 mL of THF/H₂O/acetic acid (1/1/1) and the solution was stirred for 4 hours. the reaction mixture was concentrated and the residue was dissolved in dichloromethane for further purification by column chromatography (heptane/ethyl acetate=2/3).

Deprotection of Acetyl of Sugar of Compound E (Scheme 3-v)

2.6 g (4.1 mmol) of compound E was dissolved in 50 ml of MeOH, and then 4.6 ml of 1M LiOMe (4.6 mmol Li) in 45 ml MeOH was added dropwise to this solution. After 2 h stirring at 0° C., the reaction mixture was neutralized by adding silica. After concentrating, this reaction mixture was purified continually to get hydroxyl of sugar of compound F by column chromatography (2% MeOH/ethyl acetate followed by 4% MeOH/ethyl acetate).

Activation of the Terminated-Hydroxyl Group of Compound F (Scheme 3-vi)

To a solution of 1 g of F in 60 ml of acetonitrile and 8 ml THF was added with 194 mg (2.46 mmol) of pyridine and 454 mg (2.26 mmol) of 4-Nitrophenyl chloroformate. After stirring 2 h at 0° C., the reaction mixture was concentrated and the residue was purified by column chromatography to get compound G (8% MeOH/dichloromethane).

Synthesis of DOX Prodrug DOX-mGA3 (Scheme 3-vii)

265 mg of activated compound G was dissolved in 14 ml of DMF, the above solution was added 226 mg of DOX HCl and 40 mg of triethyl amine After overnight reaction at room temperature, the reaction mixture was purified by column chromatography to get DOX prodrug DOX-mGA3 (4% methanol/ethyl acetate).

Synthesis of DOX Prodrug DOX-GA3 (Scheme 3-viii)

Dissolving DOX prodrug DOX-mGA3 in NaHCO₃ solution to hydrolyze the methyl group of the glucuronide, then adding 1M HCl to obtain DOX prodrug DOX-GA3 with —COOH group. The final compound was collected by lyophilization.

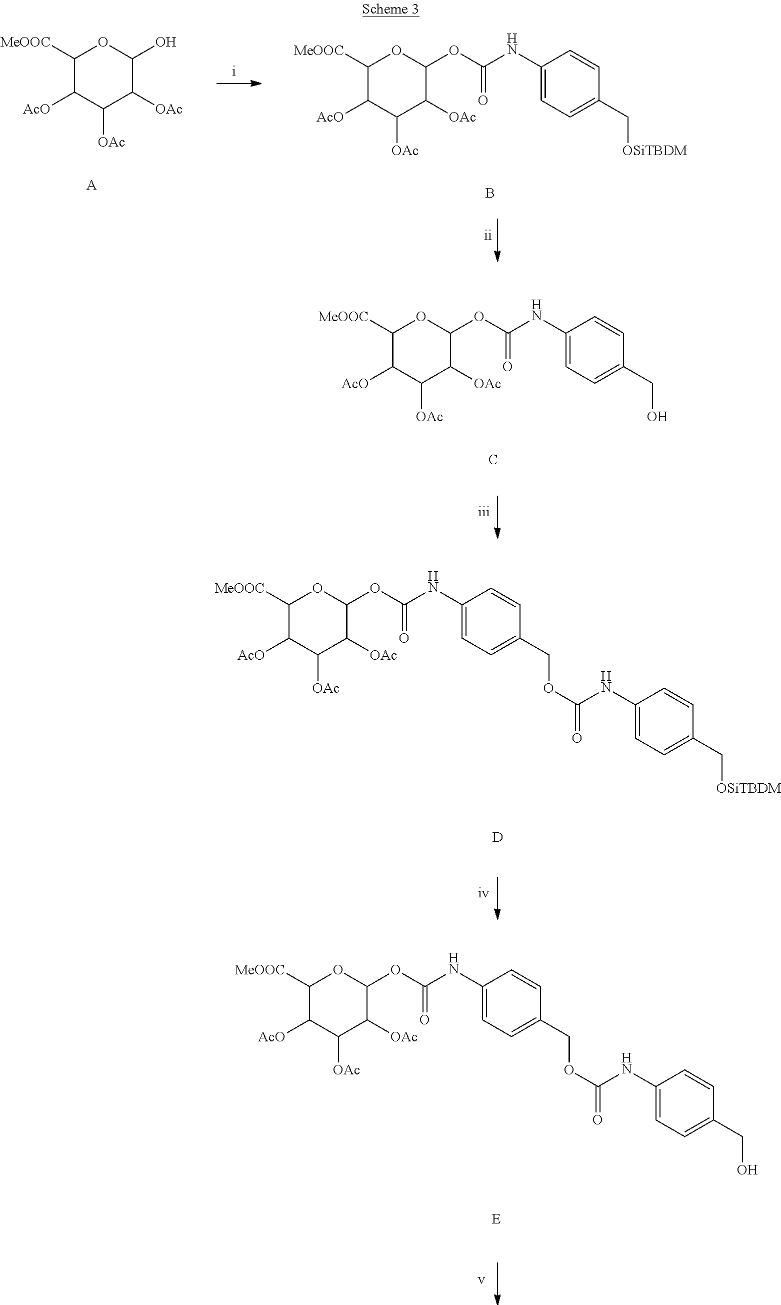

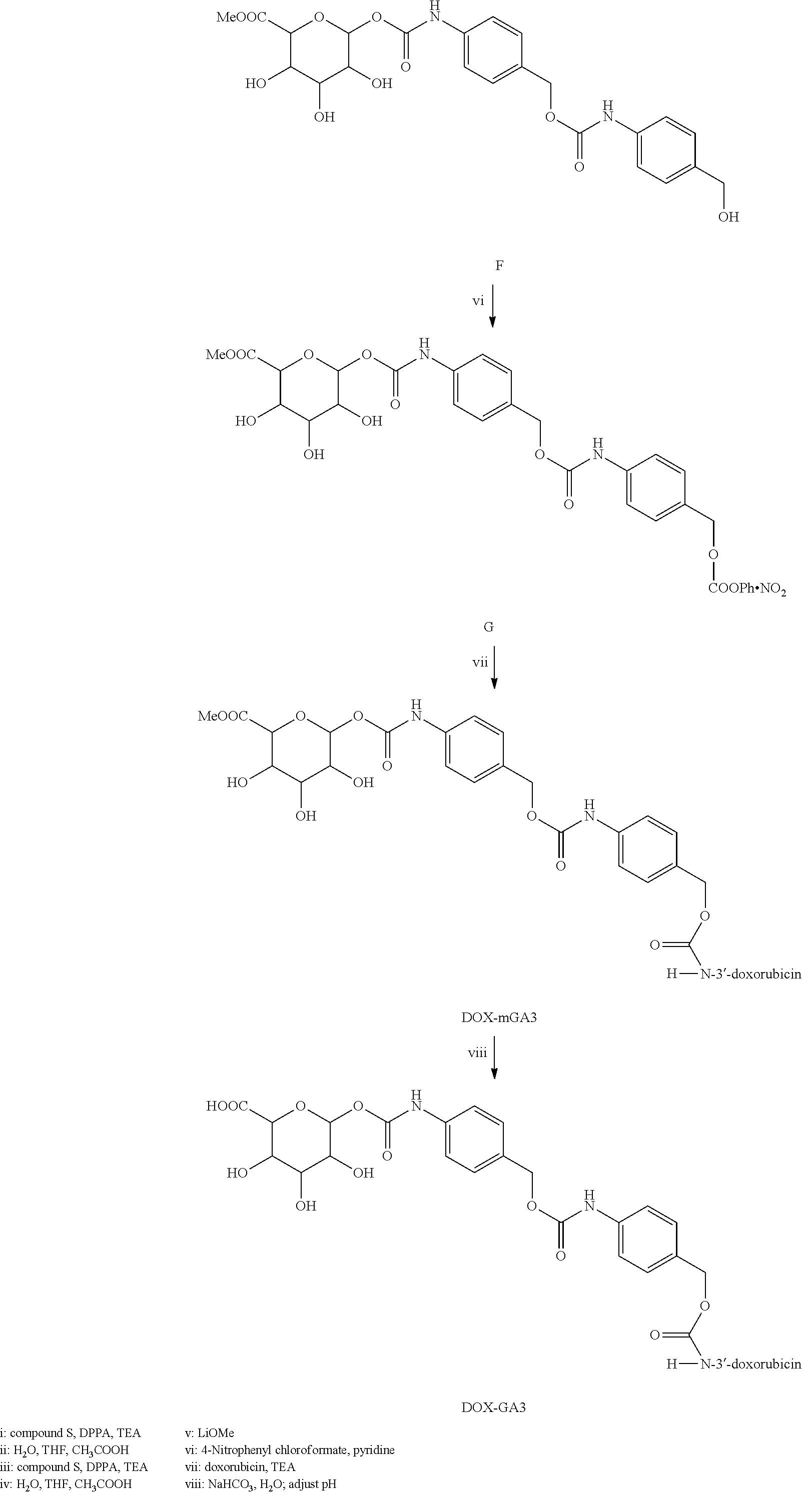

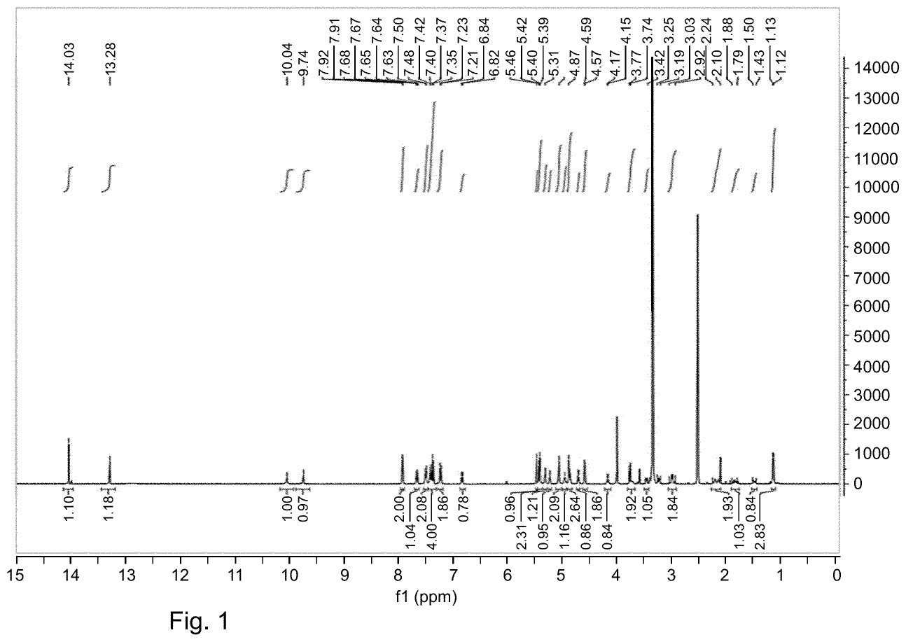

The ¹H-NMR spectrum of DOX GA3-DS is shown in FIG. 1.

Mass:
M_{fnd}=1084.28138 (M+Na)
M_{cal}=1084.28110 (M+Na)

¹H NMR (400 MHz, DMSO-d6) δ 14.04 (s, 1H, 6-OH), 13.28 (s, 1H, 11-OH), 10.03 (s, 1H, Ar-NH), 9.74 (s, 1H, Ar-NH), 7.90-7.88 (m, 2H, Ar), 7.64-7.61 (m, 1H, Ar), 7.48 (d, J=8.0 Hz, 2H, Ar), 7.44-7.29 (m, 4H, Ar), 7.22 (d, J=8.3 Hz, 2H, Ar), 6.82 (d, J=8.1 Hz, 1H, 3′—NH), 5.50-5.42 (m, 4H, Glu-2,3,4-OH, and 9-OH), 5.33 (d, J=7.9 Hz, 1H, Glu-1H) 5.21 (m, 1H, 1′-H), 5.05 (s, 2H, Ar-CH₂), 5.00-4.90 (m, 1H, 7-H), 4.87 (s, 2H, Ar-CH₂), 4.71 (d, J=8 Hz, 1H, Glu-5H) 4.57 (s, 2H, 14-CH₂), 4.15 (q, J=6.3 Hz, 1H, 5′-H), 3.99 (s, 3H, 4-OMe), 3.75 (d, J=9.1 Hz, 1H, 3′-H), 3.49-3.22 (m, 2H, Glu 2,3,4-H and 4′H partly hidden under DMSO peak), 3.04-2.92 (m, 2H, 10_{eq} and 10ₐₓ-H), 2.25-2.06 (m, 2H, 8ₐₓ and 8_{eq}-H), 1.89-1.75 (m, 1H, 2′ₐₓ-H), 1.52-1.42 (m, 1H, 2′eq-H), 1.12 (q, 3H, J=6.6 Hz, 5′-CH₃).

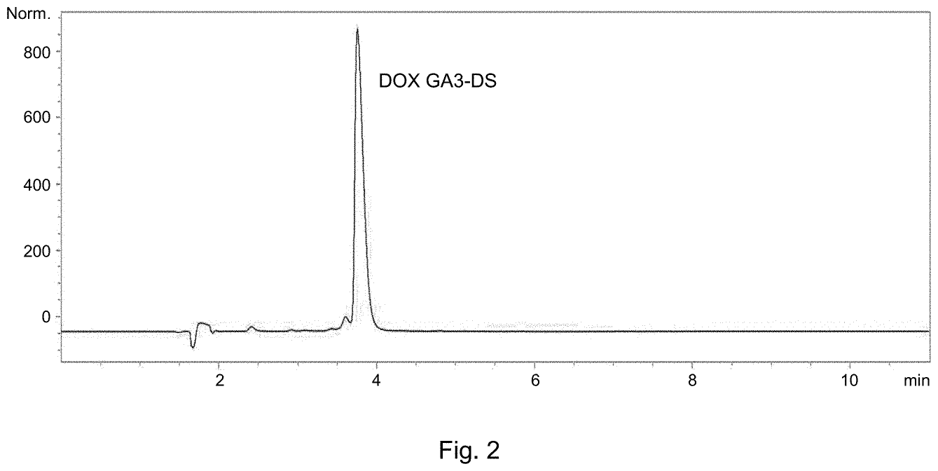

FIG. 2 shows the HPLC chromatogram of DOX GA3-DS.

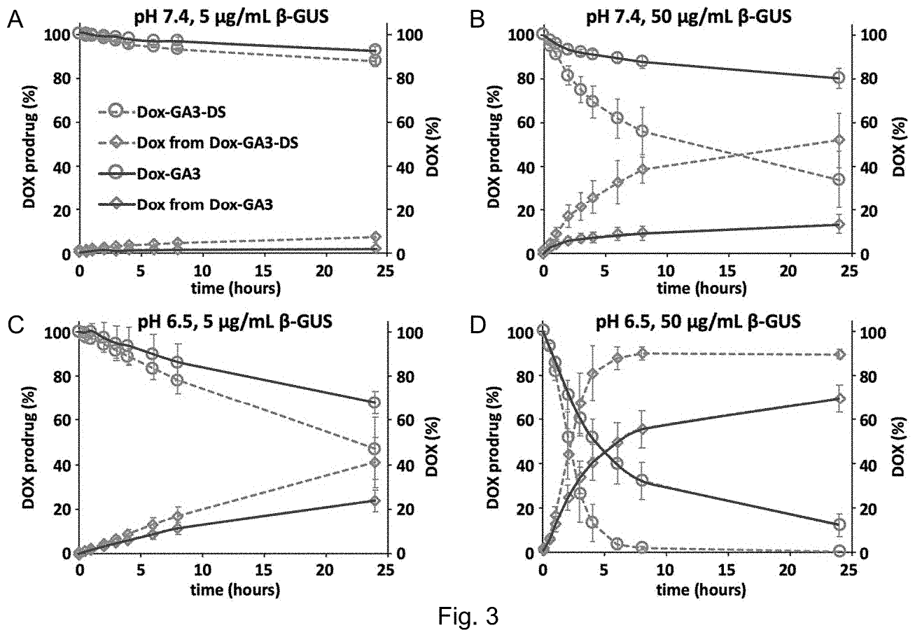

FIG. 3 shows the conversion of DOX prodrugs to DOX in the presence of beta-GUS.

Micelle Preparation and Drug Loading with DOX-GA3-DS Prodrug.

The drug-loaded mPEG-b-p(HPMAmBz) micelles were prepared using a nano-precipitation method. Firstly, different quantities of double spacer prodrug DOX-GA3 (1 mg, 5 mg or 10 mg) with 27 mg mPEG-b-p(HPMAmBz) polymer were dissolved in 1 mL THF. For DOX-GA3(—OH) with single spacer, the compound of 1 mg or 5 mg or 10 mg with 27 mg mPEG-b-p(HPMAmBz) were dissolved in a mixture of 800 μl methanol and 200 μl THF. Then the methanol-THF solution was added dropwise to 1 mL H₂O with stirring at 1000 rpm for 1 minute. The mixture was kept still at room temperature for 24 h to allow for evaporation of THF. Afterward, the DOX-GA3 prodrug-loaded micelles was filtered through 0.45 μm nylon membrane to remove non-encapsulated prodrug.

For the prodrug-loaded micelles characterization, The Z-average (Zave) size was measured by dynamic light scattering (DLS), the data is shown in the Table 1. To assess the DOX-GA3 prodrug loading content, the prodrug-loaded polymeric micelles were diluted with DMSO for at least 10 times to dissolve the micelles and the prodrugs encapsulated in the micelles, and the concentrations of the prodrugs were subsequently quantified by HPLC analysis using a Waters Acquity system. A gradient method was used, with Eluent A (H₂O with 0.1% TFA) and Eluent B (ACN with 0.1% TFA). A gradient was run with the volume fraction of eluent B increasing from 40 to 95% from 0 to 11 min and subsequently decreasing to 40% from 11 to 14 min. The injection volume was 100 μl and the detection wavelength was 485 nm. Standard solutions of the prodrugs were measured by the HPLC to generate calibration curves. The loading capacity (LC) and encapsulation efficiency (EE) of micelles can be calculated according the formula below. The results are shown in Table 1,

$$LC = \frac{c o n c e n t r ation of drug measured}{c o n c e n t r ation of ( drug measured + polymer added )} \times 100 \%$$

$$EE = \frac{c o n c e n t r ation of drug measured}{c o n c e n t r ation of drug added} \times 100 \%$$

TABLE 1
Results: final characteristics of drug-loaded micelles

| | | | | |
|---|---|---|---|---|
| feed (mg) | size (nm) | PDI | LC | EE |

DOX

| | | | | | |
|---|---|---|---|---|---|
| 1 | average | 60 | 0.08 | 2.9% | 90.9% |
| | SD | 1 | 0.02 | 0.3% | 5.2% |
| 5 | average | 72 | 0.13 | 11.6% | 78.5% |
| | SD | 1 | 0.01 | 0.9% | 4.3% |
| 10 | average | 85 | 0.19 | 16.7% | 62.9% |
| | SD | 1 | 0.01 | 0.7% | 5.2% |

DOX single spacer

| | | | | | |
|---|---|---|---|---|---|
| 1 | average | 54 | 0.18 | 2.6% | 71.4% |
| | SD | 4 | 0.02 | 0.2% | 6.7% |
| 5 | average | 55 | 0.13 | 12.6% | 78.1% |
| | SD | 3 | 0.04 | 1.1% | 7.9% |
| 10 | average | 67.57 | 0.13 | 24.6% | 88.1% |
| | SD | 1 | 0.02 | 0.49% | 2.5% |

DOX double spacer

| | | | | | |
|---|---|---|---|---|---|
| 1 | average | 47 | 0.20 | 3.3% | 92.8% |
| | SD | 3 | 0.02 | 0.3% | 9.3% |
| 5 | average | 62 | 0.23 | 16.2% | 91.7% |
| | SD | 1 | 0.02 | 1.3% | 1.2% |
| 10 | average | 61 | 0.13 | 21.5% | 75.4% |
| | SD | 1 | 0.03 | 4.2% | 18.9% |

As can be seen in Table 1 doxorubicin needs to be loaded as a hydrophobic prodrug to be able to form a uniform micellar particle formulation around 50 nm.

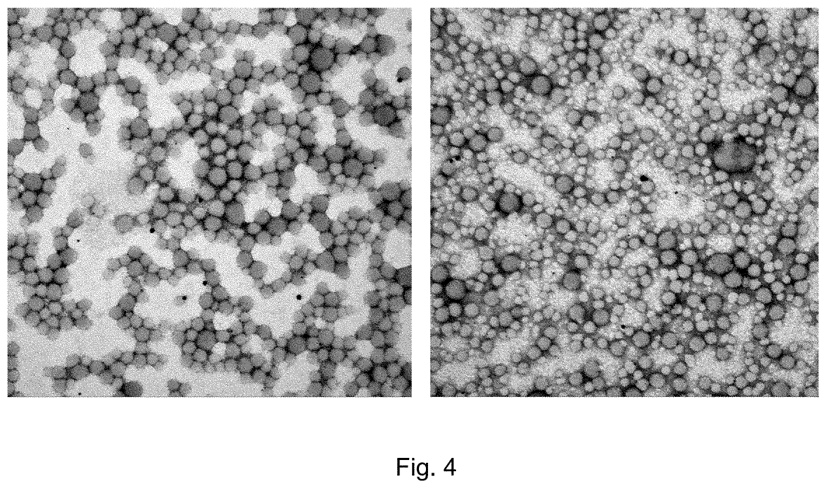

Transmission electron microscopy images were made of the micelles loaded with the single spacer prodrug of doxorubicin and compared to the micelles loaded with the double spacer doxorubicin prodrug. The results are shown in FIG. 4. As can be seen in the left panel of FIG. 4, the double spacer prodrug of doxorubicin yields a more homogeneous particle size distribution of the micelles than micelles loaded with 5 mg/mL single spacer doxorubicin prodrug (right panel).

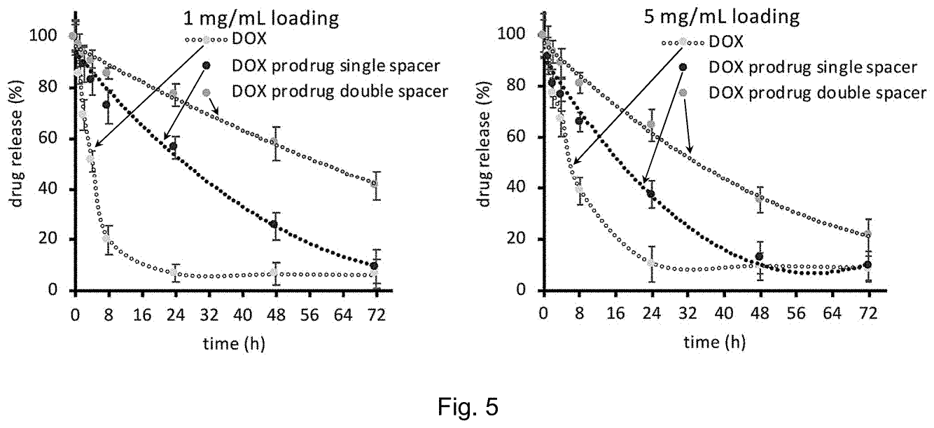

The release of doxorubicin from the micelles was studied with a dialysis method in PBS (pH=7.4) containing 0.2% v/v Tween 80 over 3 days of incubation at 37° C. At pre-set time point, aliquotes were taken from the dialysis medium and the concentration of DOX or prodrugs were analyzed by HPLC. The results are shown in FIG. 5.

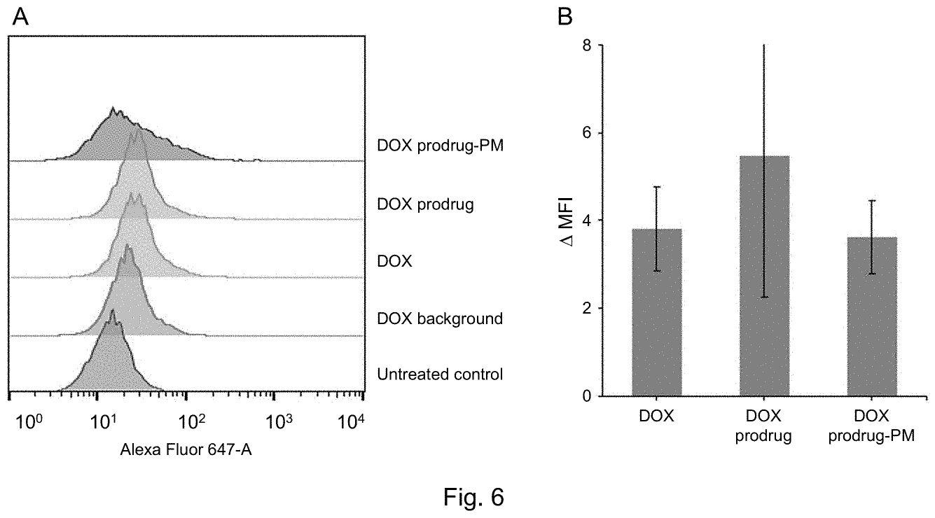

In a further experiment the ability of doxorubicin and doxorubicin double spacer prodrug and doxorubicin double spacer prodrug in polymeric micelles to induce immunogenicity of tumor cells was assessed in vitro. As one of the most studied phenotypes of immunogenic cell death, calreticulin (CRT) translation to outer surface of cancer cells is able to attract antigen presenting cells to endocytose apoptotic cancer cells. CRT translocation was measured by flow cryometry. To assess CRT translocation induced by the formulations, they were added to breast cancer 4T1 cells which were cultured overnight at 37° C. in RPMI 1640 medium containing 10% FBS and 1% penicillin. After incubation, the cells were washed with PBS and then detached. The CRT translocated to the outer surface of the cells was labeled with anti-CRT antibodies. In the flow cytometry plots in FIG. 6A, the cells treated with DOX, DOX prodrug or DOX prodrug-loaded micelles showed higher signal of CRT compared to untreated control or cells treated with DOX but not stained with the antibodies. The quantitative date in FIG. 6B indicates that the prodrug-loaded micelles and prodrug were similarly potent in inducing CRT translocation compared to DOX.

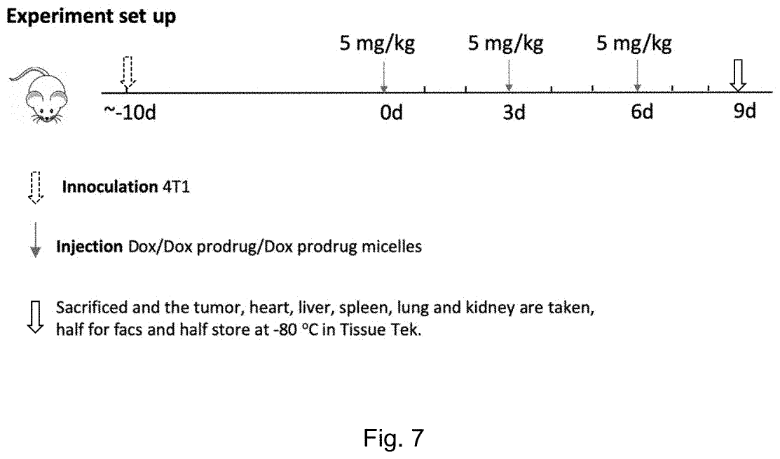

A further experiment was performed in which doxorubicin, doxorubicin double spacer prodrug and the prodrug in polymeric micelles were evaluated regarding their ability to modulate lymphocytes in the tumors in a model of triple negative breast cancer (4T1). To establish the model, 4T1 cells were inoculated in BALB/c mice. The tumor size reached ˜50-100 mm³ around 10 days after inoculation. Then the formulations were intravenously injected in the mice at 5 mg/kg in the schedule indicated in FIG. 7 (3 injection in total with 3 days intervals). At day 9 from the first injection, the mice were sacrificed the tumors were collected. The tumors were cut into two pieces, one was used for flow cytometry and the other for immunofluorescence microscopy. For flow cytometry study, the tumors were digested and the cells were suspended in PBS, which were labeled with anti-CD3 and anti-CD8 antibodies. Afterwards, the cell suspensions were washed with PBS and then analyzed by a flow cytometer. The rest of the tumor tissues were cryosectioned and stained with anti-CD4 and anti-CD8 antibodies.

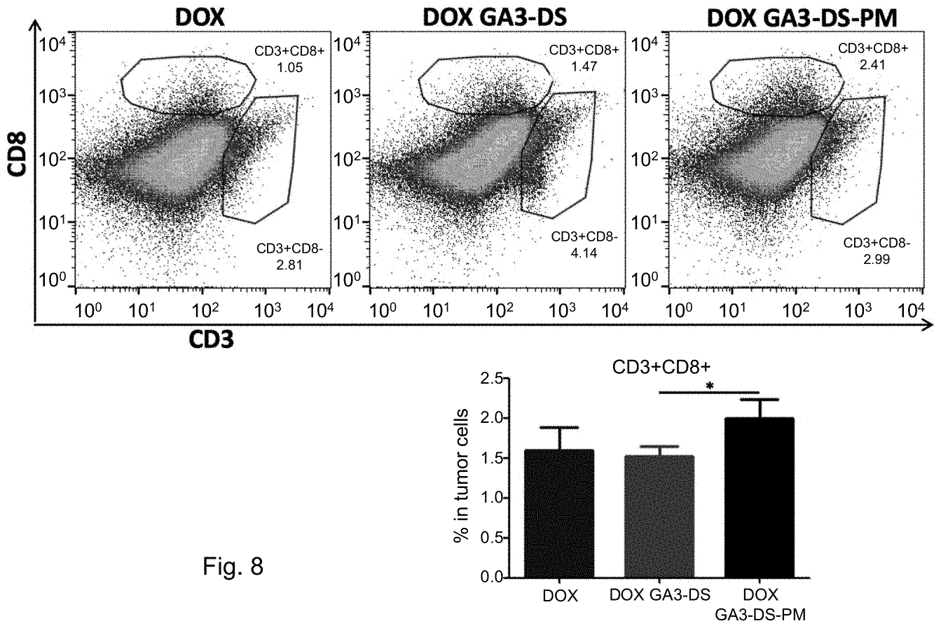

In FIG. 8, results from T cell quantification of the tumor tissues by flow cytometry were presented. Compared to tumors treated with free DOX or DOX prodrug, the tumors received prodrug-loaded micelles showed significantly higher infiltration of CD3+CD8+ T cells. The higher level of cytotoxic T cells is an important indicator of enhanced cancer immunity.

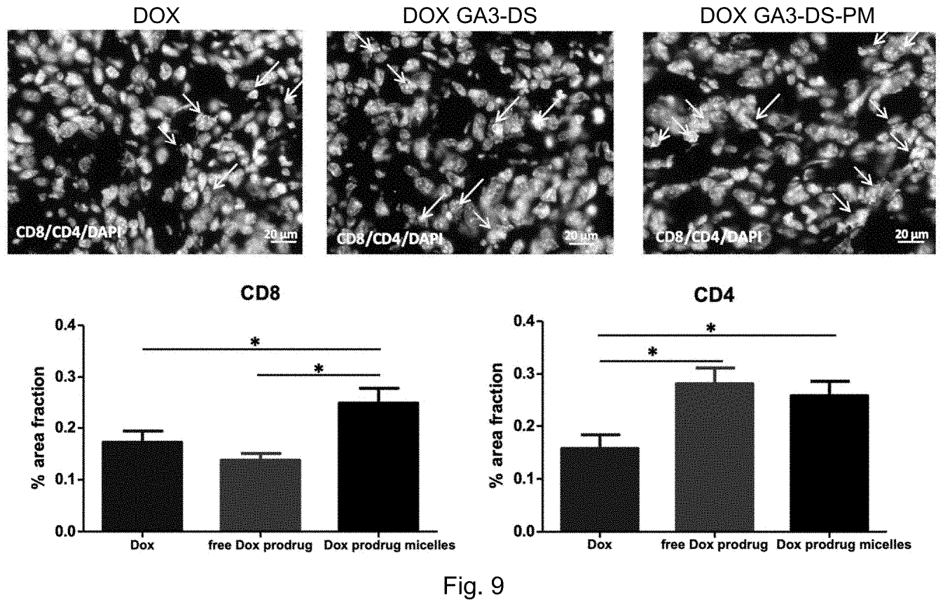

In FIG. 9, the main subpopulations of T cells (CD8+/CD4+) were identified and quantified by immunofluorescence microscopy. The amount of cytotoxic T cells (CD8+) in tumors treated with DOX prodrug-loaded micelles was significantly increased than those in DOX and DOX prodrug treated. Moreover, the CD4+ T cells in tumors were also increased by treatments with DOX prodrug or prodrug-loaded micelles compared to free DOX.

### Example 2. Synthesis and Micelle Loading of a GEM-Phe-mGA Prodrug

Prodrug Synthesis

The synthesis of the prodrug GEM-Phe-mGA was divided into 3 parts. Part 1 and 2 are similar to Example 1. and described in detail above.

Part 3: Synthesis of the Final GEM-Phe-mGA Prodrug (Scheme 4)

Synthesis of Gemcitabine-phenylalanine precursor (GEM-Phe) (based on: Bioorganic and Medicinal Chemistry 2018, 26, 5624-5630 and Molecules 2018, 23, 2608-2020)

First Part: Synthesis of Boc-Protected Phenylalanine GEM (GEM-Phe-Boc) (Scheme 4, Step ix)

1-hydroxybenzotriazole (HOBt; 68 mg, 0.50 mmol, 1 eq), N-(tert-butoxycarbonyl)-L-phenylalanine (Boc-Phe-OH; 146 mg, 0.55 mmol, 1.1 eq), 1-ethyl-3-(3-dimethylaminopropyl)carbodiimide (EDC; 125 mg, 0.65 mmol, 1.3 eq) and N,N-diisopropylethylamine (DIPEA; 65 mg, 0.5 mmol, 1 eq) were added to a solution of gemcitabine hydrochloride (GEM-HCl; 150 mg, 0.5 mmol, 1 eq) in a mixture of N,N-dimethylformamide (DMF) and dimethyl sulfoxide (DMSO) (3:1, 5 mL) at room temperature under inert atmosphere. The mixture was stirred overnight at 55° C. under inert atmosphere. After the reaction was completed, it was cooled down to room temperature and the solvent removed under reduced pressure. Brine (5 mL) was added and the mixture extracted with ethyl acetate (EtOAc; 3×10 mL). The combined organic layers were washed with a NaHCO₃ solution in water (10 mL) and brine (10 mL). The organic phase was dried over sodium sulfate and evaporated to render a white solid (GEM-Phe-Boc), which was purified by silica column chromatography (EtOAc/Hexane 4:1 with 5% MeOH). Yield: 91% (232.8 mg).

1H NMR (400 MHz, MeOD-d4): δ (in ppm) 8.39 (d, J=7.7 Hz, 1H), 7.50 (d, J=7.7 Hz, 1H), 7.35-7.19 (m, 5H), 6.35-6.24 (m, 1H), 4.51 (s, 1H), 4.36-4.32 (m, 1H), 4.04-3.94 (m, 2H), 3.89-3.80 (m, 1H), 3.18 (d, J=9.4 Hz, 1H), 2.98-2.87 (m, 1H), 1.41 (s, 9H). MS (ESI+, MeOH) for [M+Na]+, 533.181 (cal. 533.182). Reversed-phase analytical HPLC (0% MeOH/100% H₂O+0.1% TFA to 100% MeOH/0% H₂O+0.1% TFA in 30 minutes): Rt=21.3 min.

Boc-Deprotection of GEM-Phe-Boc (GEM-Phe) (Scheme 4, Step x)

To a solution of the aforementioned GEM-Phe-Boc (112 mg, 0.22 mmol) in EtOAc (6 mL), 1 M HCl (0.3 mL) were added and the solution stirred for 15 min at room temperature. The isolated precipitate was washed with EtOAc (2×10 mL) and the crude purified by column chromatography (MeOH/DCM, 1:9) to afford the desired compound GEM-Phe. Yield: 22% (20 mg).

1H NMR (400 MHz, MeOD-d4): δ (in ppm) 7.75 (d, J=7.6 Hz, 1H), 7.27-7.13 (m, 5H), 6.20-6.12 (m, 1H), 5.90 (d, J=7.6 Hz, 1H), 4.95 (dd, J=8.5, 6.1 Hz, 1H), 4.21 (td, J=12.1, 8.3 Hz, 1H), 3.96-3.80 (m, 2H), 3.78-3.64 (m, 1H), 3.18 (dd, J=13.9, 6.1 Hz, 1H), 2.95 (dd, J=13.9, 8.5 Hz, 1H). MS (ESI+, MeOH) for [M+H]+, 411.147 (cal. 411.148); for [M+Na]+, 433.128 (cal. 433.130). Reversed-phase analytical HPLC (0% MeOH/100% H₂O+0.1% TFA to 100% MeOH/0% H₂O+0.1% TFA in 30 minutes): Rt=14.1 min.

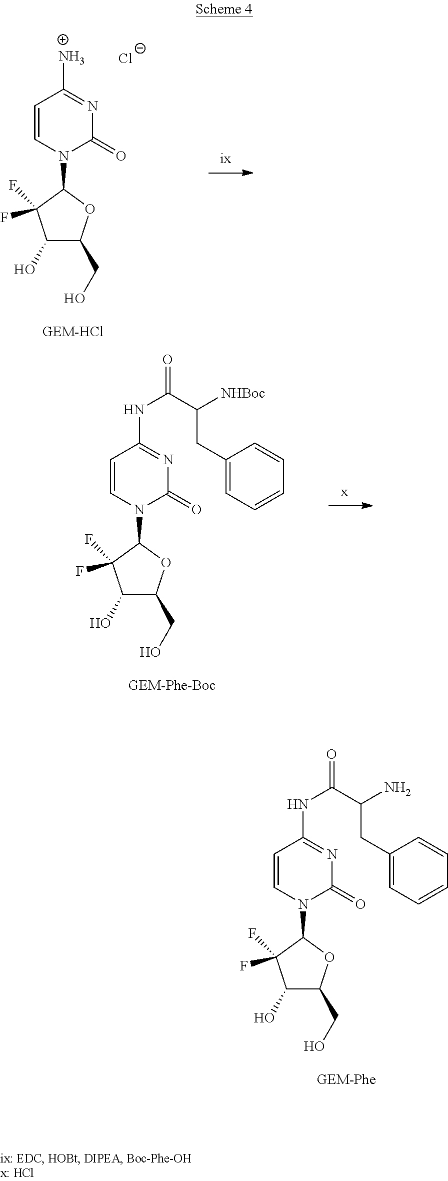

Second Part: Synthesis of GEMCITABINE Prodrug GEM-Phe-mGA3 (Scheme 5)

Activation of the Benzyl Alcohol of Compound C: Compound H (Scheme 5, Step xi)

Compound C (150 mg, 0.42 mmol, 1 eq) was dissolved in a mixture of ACN (15 mL) and THF (2 mL). After cooling the mixture down to 0° C., pyridine (44 μL, 0.55 mmol, 1.3 eq) and 4-nitrophenyl chloroformate (Cl—COOPhNO₂, 102 mg, 0.50 mmol, 1.2 eq) were added. The reaction was stirred for 2 hours and the crude was purified by silica column chromatography (8% MeOH in DCM) to afford compound H. Yield: 37% (80.5 mg).

1H NMR (600 MHz, MeOD-d4): δ (in ppm) 8.39-8.23 (m, 2H), 7.58-7.36 (m, 6H), 5.52 (d, J=8.1 Hz, 1H), 5.25 (s, 2H), 4.02 (d, J=9.7 Hz, 1H), 3.76 (s, 3H), 3.58 (t, J=9.3 Hz, 1H), 3.50 (t, J=9.0 Hz, 1H), 3.44 (dd, J=9.1 Hz, 1H). Reversed-phase analytical HPLC (0% MeOH/100% H₂O+0.1% TFA to 100% MeOH/0% H₂O+0.1% TFA in 30 minutes): Rt=20.7 min.

Synthesis of GEM-Prodrug GEM-Phe-mGA3 (Scheme 5, Step xii)

Compound H (45 mg, 0.086 mmol, 1.2 eq) was dissolved in anhydrous DMF (2 mL) under nitrogen atmosphere. GEM-Phe (30 mg, 0.072 mmol, 1 eq), TEA (10 μL, 0.072 mmol, 1 eq), HOBt (10 mg, 0.072 mmol, 1 eq) and DIPEA (13 μL, 0.072 mmol, 1 eq) were added to this solution and the mixture kept under stirring overnight at room temperature. The DMF was removed and the crude was purified by silica column chromatography (10% MeOH in DCM) to yield GEM-Phe-mGA3. Yield: 41% (23.6 mg).

1H NMR (400 MHz, MeOD-d4): δ (in ppm) 8.36 (d, J=7.6 Hz, 1H), 7.82 (d, J=8.4 Hz, 1H), 7.71 (d, J=8.3 Hz, 1H), 7.30-7.08 (m, 8H), 6.27-6.23 (m, 1H), 5.50 (d, J=8.1 Hz, 1H), 4.96 (m, 2H), 4.56 (dd, J=9.0, 5.7 Hz, 1H), 4.35-4.26 (m, 1H), 3.98 (d, J=9.7 Hz, 2H), 3.81 (dd, J=12.9, 3.2 Hz, 1H), 3.76 (s, 3H), 3.56 (t, J=9.3 Hz, 1H), 3.49 (t, J=9.0 Hz, 1H), 3.46-3.40 (t, J=9.1 Hz, 1H), 3.16-3.08 (m, 1H), 2.96-2.89 (m, 1H). MS (ESI+, MeOH) for [M+Na]+, 816.22 (cal. 816.2152). MS (ESI−, MeOH) for [M+Cl]−, 828.1960 (cal. 828.1943). Reversed-phase analytical HPLC (0% MeOH/100% H₂O+0.1% TFA to 100% MeOH/0% H₂O+0.1% TFA in 30 minutes): Rt=17.9 min.

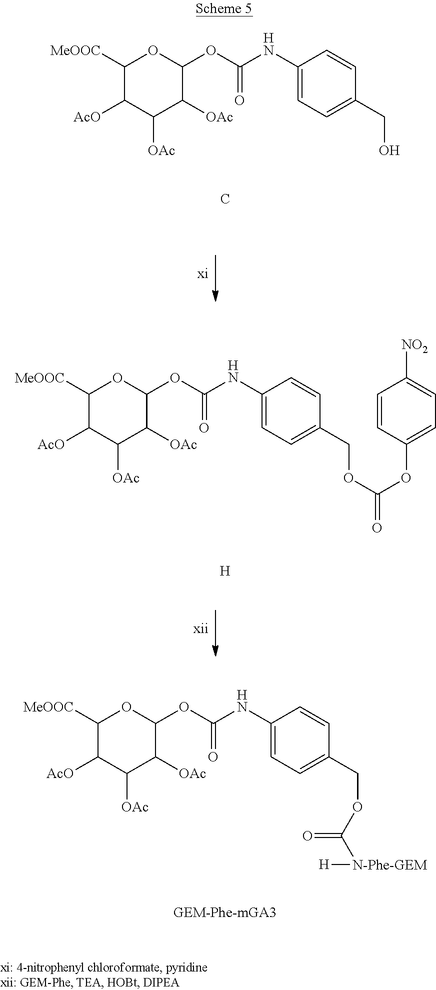

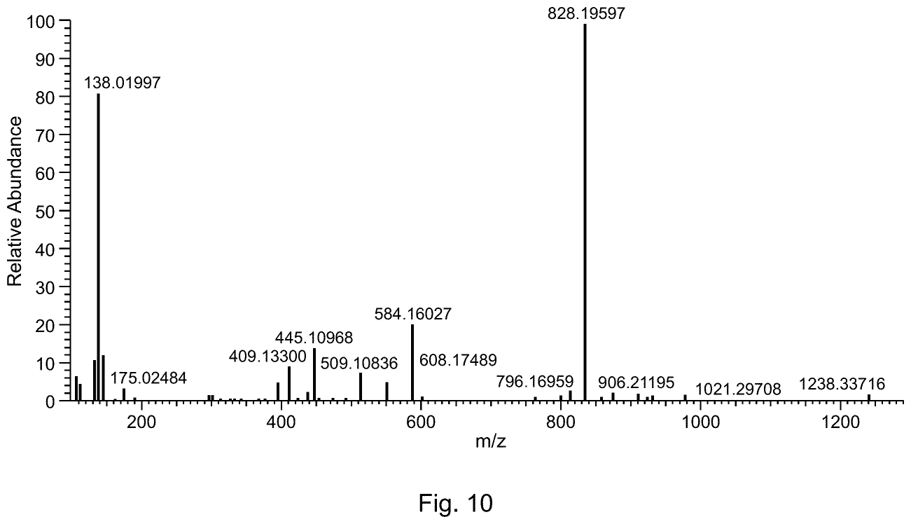

The mass spectrum of GEM-Phe-mGA3 is shown in FIG. 10.

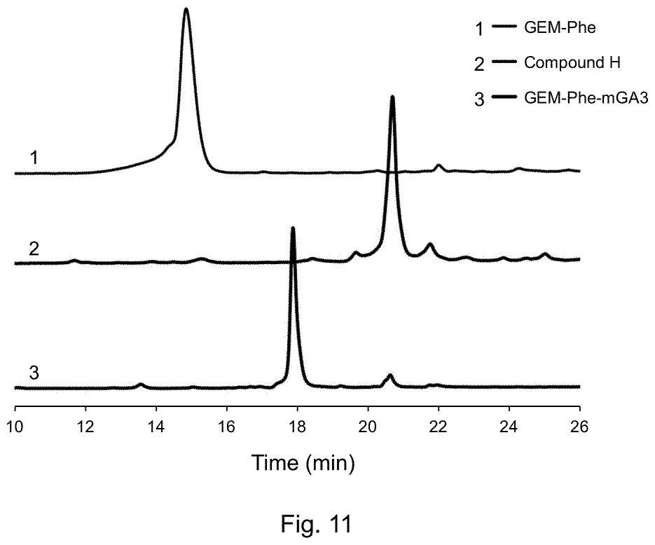

FIG. 11 shows the HPLC chromatogram of GEM-Phe-mGA3.

Micelle Preparation and Drug Loading with GEM-Phe-mGA3 Prodrug

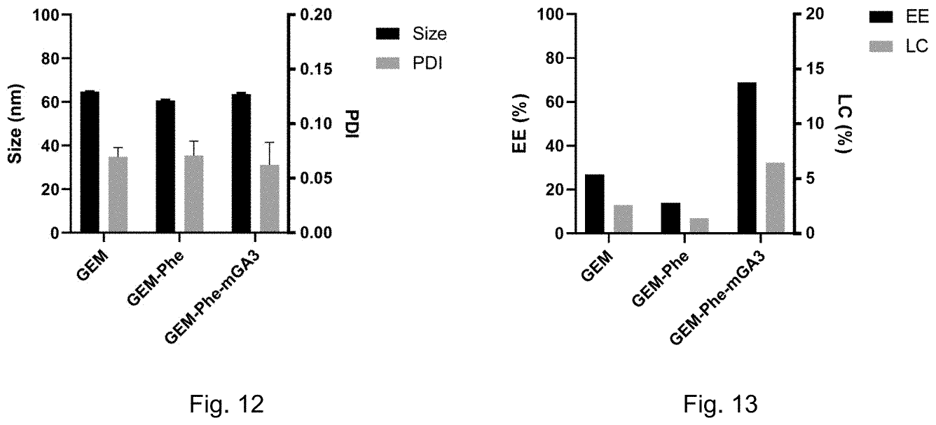

The drug-loaded mPEG-b-p(HPMAmBz) micelles were prepared using a nano-precipitation method. Firstly, single spacer phenylalanine GEM prodrug GEM-Phe-mGA3 (1 mg), GEM (1 mg) and GEM-Phe (1 mg) with 10 mg mPEG-b-p(HPMAmBz) polymer (1:10; drug:polymer ratio) were dissolved in 1 mL THF containing 10% MeOH. Then the THF/MeOH solution was added dropwise to 1 mL H₂O with stirring at 1000 rpm for 1 minute. The mixture was kept still at room temperature for 24 h to allow for evaporation of THF and MeOH. Afterwards, the GEM, GEM-Phe and GEM-Phe-mGA3 prodrug-loaded micelles were filtered through 0.2 μm nylon membrane to remove non-encapsulated prodrug. For the prodrug-loaded micelles characterization, the Z-average (Zave) size was measured by dynamic light scattering (DLS), the data is shown in FIG. 12. To assess the GEM-Phe prodrug and GEM-mGA3 prodrug loading content, the prodrug-loaded polymeric micelles were diluted with MeOH for at least 10 times to dissolve the micelles and the prodrugs encapsulated in the micelles, and the concentration of the prodrugs were subsequently quantified by reversed-phase HPLC analysis using a C18 column. In the case of GEM, between 10-20% of DMSO was added. A gradient method was used, with Eluent A (H₂O with 0.1% TFA) and Eluent B (MeOH with 0.1% TFA). A gradient was run with the volume fraction of eluent B increasing from 0 to 100% from 0 to 30 min. The injection volume was 100 μl and the detection wavelength was 260 nm. The loading capacity (LC) and encapsulation efficiency (EE) of micelles can be calculated according to the formula below. The results are shown in FIG. 13.

LC=(concentration of drug measured)/(concentration of (drug measured+polymer added))×100%

EE=(concentration of drug measured)/(concentration of drug added)×100%

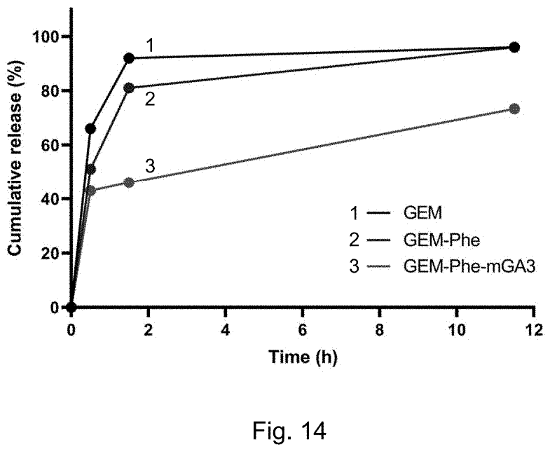

The release of the compounds from the micelles was studied with a dialysis method in PBS (pH 7.4) containing 30 mg/mL of BSA over 12 h incubation at 37° C. At pre-set time point, aliquots were taken from the dialysis medium and the concentration of GEM or GEM prodrugs were analyzed by HPLC. The results are shown in FIG. 14.

### Example 3 Synthesis Bifurcated Glucuronide Structure (for Compounds of Formula (III) of the Invention)

The bifurcated glucuronide structure can be synthesized as described in Tranoy-Opalinski et al. 2008, for instance as shown in scheme 6 below.

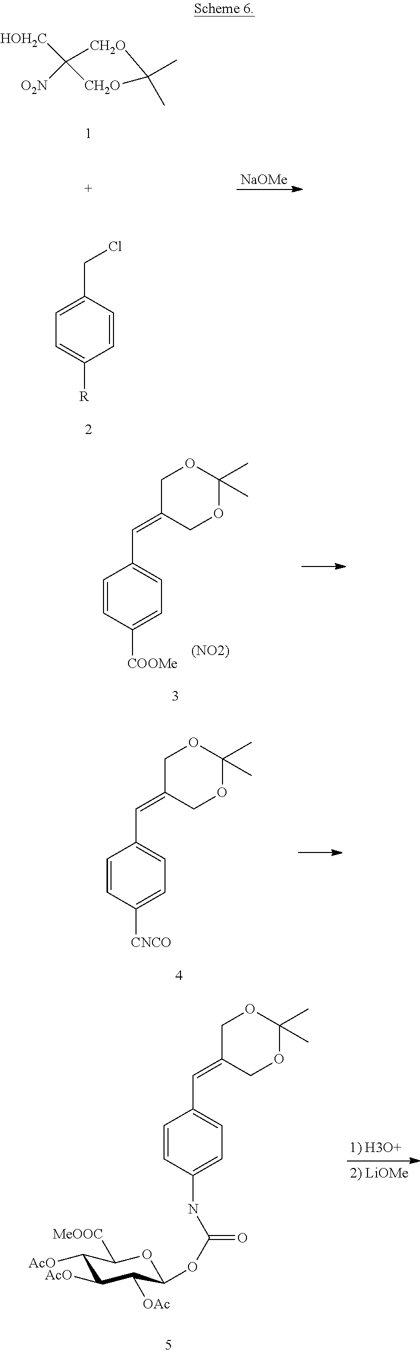

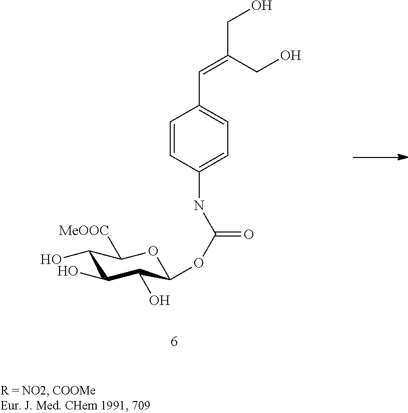

### REFERENCES

De Graaf et al. Biochem Pharmacol 2004 Dec. 1; 68(11):2273-81.doi:10.1016/j.bcp.2004.08.004.
De Groot et al. J Org Chem 2001 Dec. 28; 66(26):8815-30. doi: 10.1021/jo0158884.
Houba et al. Br J Cancer. 1998 December; 78(12):1600-6. doi: 10.1038/bjc.1998.729.
Houba et al. Br J Cancer. 2001 February; 84(4):550-7. doi: 10.1054/bjoc.2000.1640.
Leenders et al. Bioorg Med Chem. 1999 August; 7(8):1597-610. doi: 10.1016/s0968-0896(99)00095-4.
D. Neradovic et al. Macromolecules, 2001, 34 (22), pp 7589-7591
Ruiz-Hernandez et al. Polym Chem. 2014 Mar. 7; (5):1674-1681. doi: 10.1039/C3PY01097J.
Shi et al., Biomacromolecules 2013, 14, 1826-1837
Talleli et al. Bioconjug Chem 2011 Dec. 21; 22(12):2519-30. doi: 10.1021/bc2003499.
Tranoy-Opalinski et al. Anticancer Agents Med Chem. 2008 August; 8(6):618-37.

1. A polymeric micelle comprising a block copolymer comprising a polyethylene glycol (PEG) hydrophilic block and a hydrophobic block, and a compound according to formula (I) or formula (III) encapsulated within said polymeric micelle 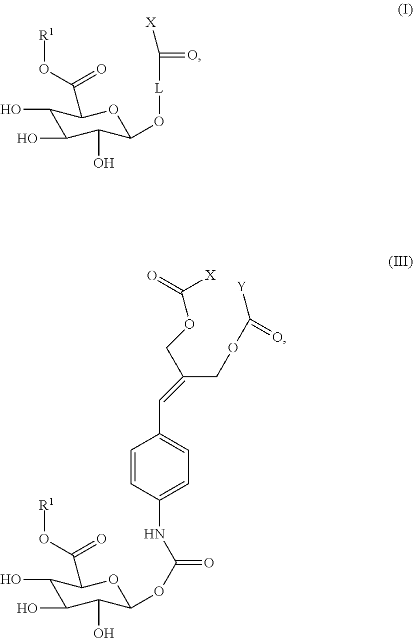
wherein
X and Y are independently a hydrophilic drug molecule,
R¹ is H or —(CH₂)ₚ—CH₃, wherein p is 0, 1, 2 or 3, or R¹ is a drug molecule and
L is a spacer molecule comprising
2 to 6 aromatic rings oriented in a linear manner, or
1-5 aromatic rings oriented in a linear manner and one or more aromatic rings as a pendant side group or
1-5 aromatic rings and one or more double carbon-carbon bonds oriented in a linear manner,
wherein said rings are each optionally and independently substituted with at least one halogen atom, hydroxyl or alkoxy group, and/or at least one (C1-4)-alkyl.

2. The polymeric micelle according to claim 1 of formula (I) 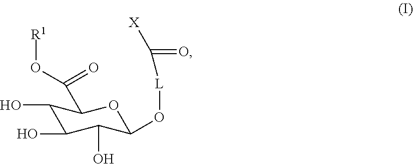
wherein
X is a hydrophilic drug molecule,
R¹ is H or —(CH₂)ₚ—CH₃, wherein p is 0, 1, 2 or 3, or R¹ is a drug molecule, and
L is a spacer molecule comprising 2 to 6 aromatic rings, wherein said rings are each optionally and independently substituted with at least one halogen atom and/or at least one (C1-4)-alkyl.

3. The polymeric micelle according to claim 1 of formula (III) 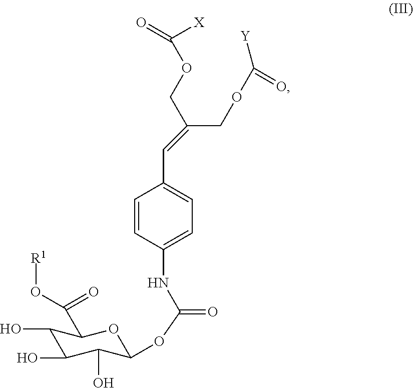
wherein
X and Y are independently a hydrophilic drug molecule, and
R¹ is H or —(CH₂)ₚ—CH₃, wherein p is 0, 1, 2 or 3, or R¹ is a drug molecule.

4. The polymeric micelle according to claim 1 wherein L comprises 2 to 6 aryl rings.

5. The polymeric micelle according to claim 1 wherein the compound is selected from the group consisting of formula's (Ia), (Ib), (Ic), (Id), (Ie), (If), (Ig), (Ih), (Ii) and (III). 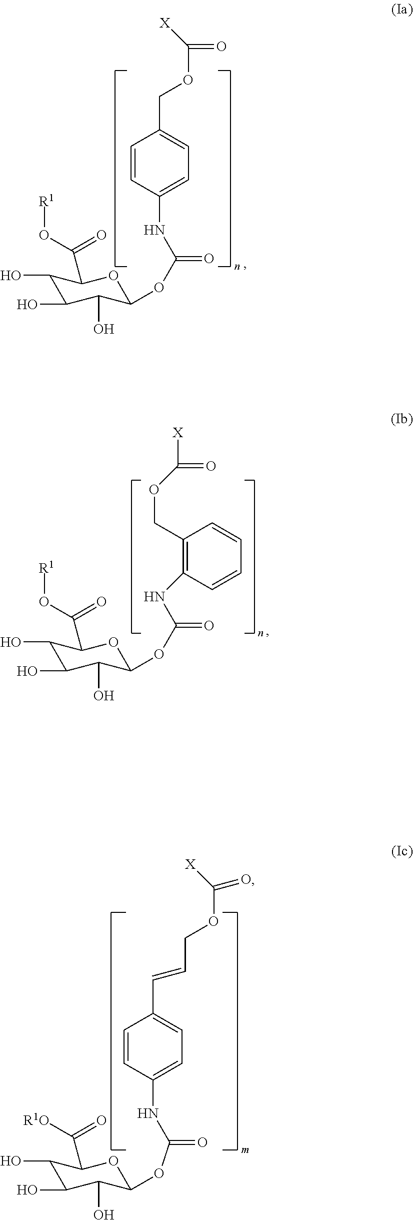 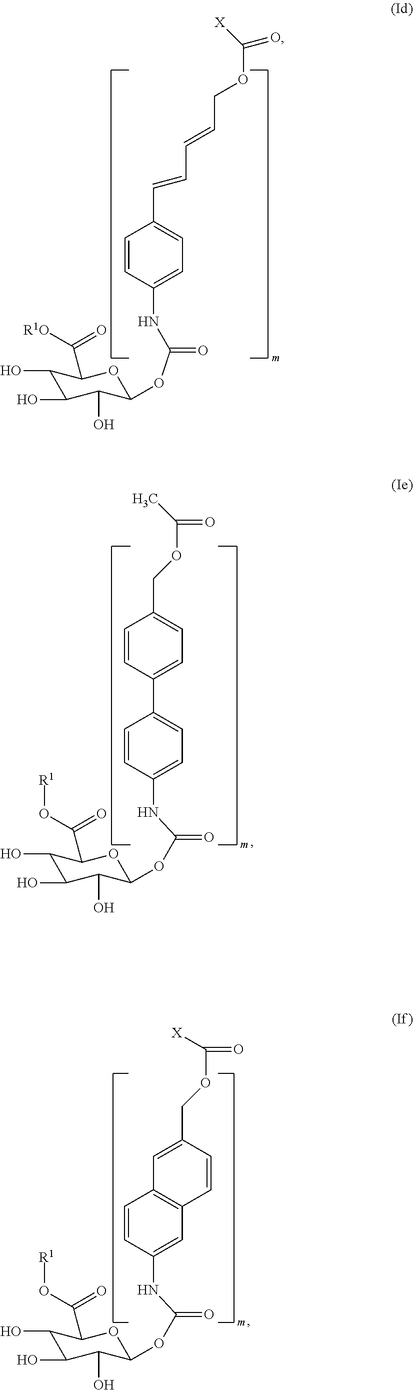 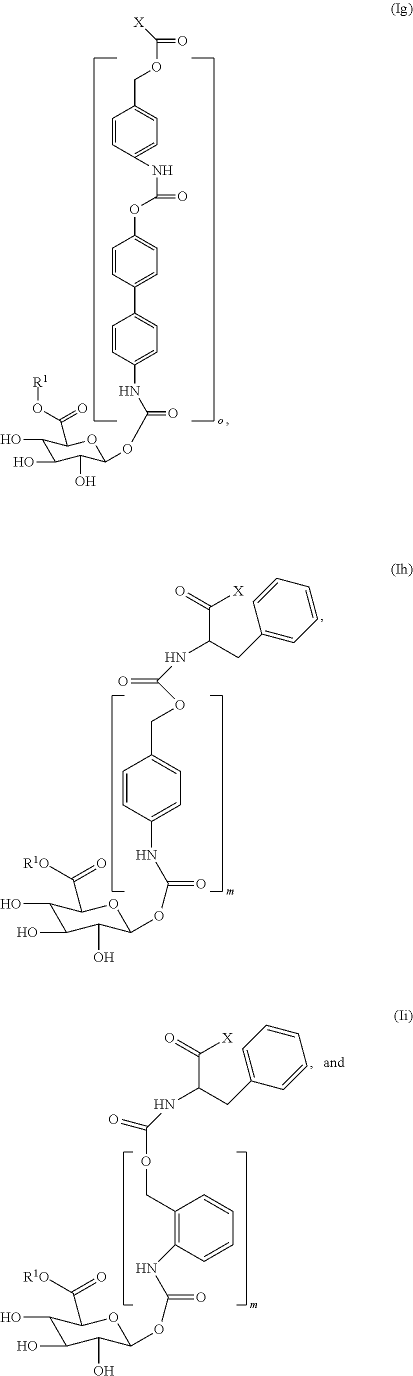 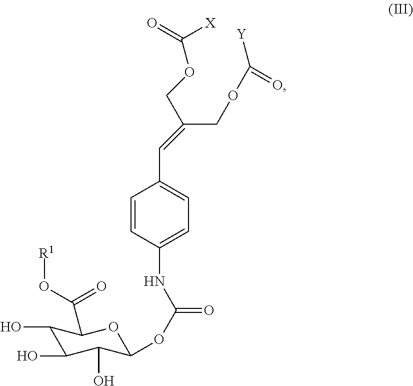
wherein R¹ is H or —(CH₂)ₚ—CH₃, wherein p is 0, 1, 2 or 3, or R¹ is a drug molecule, and wherein n is 2 to 6, m is 1 to 3, and o is 1 or 2.

6. The polymeric micelle according to claim 1, wherein R¹ is H.

7. The polymeric micelle according to claim 1, wherein R¹ is a platinum compound.

8. The polymeric micelle according to claim 1, wherein each hydrophilic drug molecule is selected from the group consisting of an anticancer drug, an antifungal drug, an antibiotic drug and a combination thereof.

9. The polymeric micelle according to claim 1, wherein said hydrophilic drug molecule is selected from the group consisting of anthracyclines, nucleoside or deoxycytidine analogues, topoisomerase I inhibitors, nitrogen mustard alkylating agents, immunomodulators, adjuvants, P-glycoprotein drug efflux pump inhibitors, taxanes, anticancer peptides drug molecules, anticancer nucleic acid compounds and platinum compounds; the hydrophilic drug molecule.

10. The polymeric micelle according to claim 1 wherein at least part of the hydrophobic block contains an aromatic side group.

11. A pharmaceutical composition comprising a polymeric micelle according to claim 1 and at least one pharmaceutically acceptable carrier, diluent or excipient.

12. (canceled)

13. A method for the treatment or prevention of cancer or infection in a subject in need thereof, comprising administering to the subject a therapeutically effective amount of a polymeric micelle according to claim 1.

14. (canceled)

15. A method of immunotherapy in a subject in need thereof, the method comprising administering to the subject a therapeutically effective amount of a polymeric micelle according to claim 1.

16. (canceled)

17. The method according to claim 15 for enhancing efficacy of immunotherapy in a subject suffering from cancer and being treated with said immunotherapy, the method comprising administering to the subject a therapeutically effective amount of a polymeric micelle according to claim 1.

18. (canceled)

19. The polymeric micelle according to claim 4 wherein said aryl rings are independently selected from the group consisting of phenyl rings, naphthyl, biphenyl groups, and combinations thereof.

20. The polymeric micelle according to claim 7, wherein the platinum compound is selected from the group consisting of cisplatin, oxaliplatin and carboplatin.

21. The polymeric micelle according to claim 9, wherein the hydrophilic drug molecule is selected from the group consisting of doxorubicin, daunorubicin, irinotecan and gemcitabine.

22. The polymeric micelle according to claim 10 wherein the aromatic side group is an aryl or heteroaryl.

23. The polymeric micelle according to claim 22 wherein the aromatic side group is selected from the group consisting of benzyl, phenyl and naphthyl.

24. The method according to claim 17, wherein said immunotherapy is antibody-based immunotherapy.
